SEQUENCE LISTING (36 sequences)

SEQ ID NO: 1 - DNA, 31 bp, Artificial
  PCR primer for SDM
ggtccattcactagtctcattcccttttcag

SEQ ID NO: 2 - DNA, 31 bp, Artificial
  PCR primer for SDM
ctgaaaagggaatgagactagtgaatggacc

SEQ ID NO: 3 - DNA, 27 bp, Artificial
  PCR primer for spectinomycin cassette
actagtgaggaggatatatttgaatac

SEQ ID NO: 4 - DNA, 37 bp, Artificial
  PCR primer for spectinomycin cassette
agatctttataatttttttaatctgttatttaaatag

SEQ ID NO: 5 - DNA, 28 bp, Artificial
  PCR primer for tetracycline cassette
actagtgaatacatcctattcacaatcg

SEQ ID NO: 6 - DNA, 27 bp, Artificial
  PCR primer for tetracycline cassette
agatctttagaaatccctttgagaatg

SEQ ID NO: 7 - DNA, 30 bp, Artificial
  PCR primer for erythromycin cassette
actagtgaacgagaaaaatataaaacacag

SEQ ID NO: 8 - DNA, 34 bp, Artificial
  PCR primer for Erythromycin cassette
agatctttacttattaaataatttatagctattg

SEQ ID NO: 9 - DNA, 36 bp, Artificial
  Short HIS tag
atgagaggatcgcatcaccatcaccatcactcgcga

SEQ ID NO: 10 - DNA, 36 bp, Artificial
  Short HIS tag
tcgcgagtgatggtgatggtgatgcgatcctctcat

SEQ ID NO: 11 - DNA, 48 bp, Artificial
  Long HIS tag
ggatccgcatgcgagctcggtgccggccgacctgcagccaagaggcct

SEQ ID NO: 12 - DNA, 48 bp, Artificial
  Long HIS tag
aggcctcttggctgcaggtcggccggcaccgagctcgcatgcggatcc

SEQ ID NO: 13 - DNA, 26 bp, Artificial
  PCR primer for pLdh promoter
catccgctatatattaacgtgggtgc

SEQ ID NO: 14 - DNA, 32 bp, Artificial
  PCR primer for pLdh promoter
ccaccgttgtttttcatatgattcatcctccc

SEQ ID NO: 15 - DNA, 29 bp, Artificial
  PCR primer for pLdh promoter
cccgggttcatcctccctcaatataatgc

SEQ ID NO: 16 - DNA, 26 bp, Artificial
  PCR primer for pSec promoter
cctagggtgcgatgagtttcgtcttc

SEQ ID NO: 17 - DNA, 27 bp, Artificial
  PCR primer for pSec promoter
catatgccttaagattcctccttcaac

SEQ ID NO: 18 - DNA, 20 bp, Artificial
  PCR primer for pGal promoter
gtgattttactgtatccttc

SEQ ID NO: 19 - DNA, 28 bp, Artificial
  PCR primer for pGal promoter
cccgggtattccccctagctaattttcg

SEQ ID NO: 20 - DNA, 28 bp, Artificial
  PCR primer for B-galactosidase gene
atgaatgtgttatcctcaatttgttacg

SEQ ID NO: 21 - DNA, 25 bp, Artificial
  PCR primer for B-galactosidase gene
ctaaaccttcccggcttcatcatgc

SEQ ID NO: 22 - DNA, 24 bp, Artificial
  PCR primer for Bt 1.3.99.2 gene
catatggaggcggagtatatgaac

SEQ ID NO: 23 - DNA, 36 bp, Artificial
  PCR primer for Bt 1.3.99.2 gene
ctcgagttaggtacctaaagcacggctaataacaag

SEQ ID NO: 24 - DNA, 26 bp, Artificial
  PCR primer for Bt 1.3.99.2 (both) genes
atggaggcggagtatatgaactttcg

SEQ ID NO: 25 - DNA, 27 bp, Artificial
  PCR primer for Bt 1.3.99.2 (both) genes
ctacttcgttaacatacgagaaattac

SEQ ID NO: 26 - DNA, 28 bp, Artificial
  PCR primer for Bt FeS gene
atgaatagcttactgatcattaattggc

SEQ ID NO: 27 - DNA, 28 bp, Artificial
  PCR primer for Bt FeS gene
cccgggtgcagattacatcgcttctttc

SEQ ID NO: 28 - DNA, 25 bp, Artificial
  PCR primer for Tats 1.3.99.2 + EtfAB genes
atggacttttcattaacaaaggagc

SEQ ID NO: 29 - DNA, 33 bp, Artificial
  PCR primer for Tats 1.3.99.2 + EtfAB genes
cccgggacactcctttttatttttattaaggtc

SEQ ID NO: 30 - DNA, 23 bp, Artificial
  PCR primer for Ec 1.2.1.10 gene
catatggctgttactaatgtcgc

SEQ ID NO: 31 - DNA, 45 bp, Artificial
  PCR primer for Ec 1.2.1.10 gene
gagctcggatccttacccgggagcggattttttcgcttttttctc

SEQ ID NO: 32 - DNA, 28 bp, Artificial
  PCR primer for Bt 1.2.1.10 gene
catatgatgagaagcttcaaacctgatg

SEQ ID NO: 33 - DNA, 51 bp, Artificial
  PCR primer for Bt 1.2.1.10 gene
gagctcggatccttacccgggttcacctttatatgcttccatataaatttc

SEQ ID NO: 34 - DNA, 42 bp, Artificial
  PCR primer for Tats butanol operon
ctcgagggatggattttaataatgttttattaaataaggatg

SEQ ID NO: 35 - DNA, 33 bp, Artificial
  PCR primer for Tats butanol operon
gttaacctatctttcgacaaccattgctattcc

SEQ ID NO: 36 - DNA, 5863 bp, Thermoanaerobacterium thermosaccharolyticum
atggattttaataatgttttattaaataaggatgatgggatagctctcatcattataaatcgtccaaaggctttaaatgcattaaactatgagacactaaaagagttagatagtgtgcttgatatagttgaaaatgataaagagataaaagttttaattataactggcagcggtgaaaaaaccttcgttgcaggtgctgatatagctgagatgagtaatatgacaccacttgaagcgaagaagttctctctttatggacagaaagtatttaggaagatagaaatgctaagtaagcctgttatagcagcggtaaatggttttgcacttggtggtggatgcgagctttctatggcatgtgacatacgtattgcaagtaaaaatgcaaaatttggtcaacctgaagtaggacttggaataatacctggcttttcaggaactcaaagattaccacgtcttataggcacttctaaagctaaagagcttattttcacaggtgacatgataaattctgatgaagcatataaaataggccttatatctaaagttgttgaactatctgatctcattgaagaagcaaaaaaactcgcgaaaaaaatgatgtcaaaaagtcaaatagcaatttctctagcaaaggaagcaataaataagggaatggaaacagacttagatacaggcaatactatagaagctgagaaattttccttatgttttacaacagatgatcaaaaagaaggtatgattgcgttttctgaaaagagggcgcctaaatttggcaaataatatcaatttttgttacaaaattatcaataaggaggcaattttatggacttttcattaacaaaggagcaagaaatggtaaggcgtgttgtgagagaattcgctgaaaaagaagttgctcctaaagcaaaagaaatagatatcacagaagagtttccatgggatacagtaagaaaaatggctcaaaacgatatgatgggtattccttatccagaagagtatggtggagcaggtggagattacttgagttatatcatagctgttgaagagatatcaagagcttgtgctacgactggagtaattttatctgctcatacttcattgggaagttttccaatatatcaatggggaacagaagaacaaaaaagaaaatatctagtgccacttgcaaaaggtgaaaaattgggcgcttttggccttacagaacctaacgcaggtacagatgcagctggacagcagacaactgcagtattagatggtgatcactacgtattaaacggctcaatatttattacaaacggaggaaaagctgacatatatataatctttgcaatgacagacaaatcaaaaggcacaagaggcattagtgcatttatagttgagaaagattttccgggttttagcattggcaaaattgaagaaaaaatgggtataagagcttcatcaactgccgaacttgtgtttgaagattgtattgtaccaaaagaaaatttacttggtaaagaaggagaaggttttaaaattgcgatggctacactagatggtggaagaataggaatagcagcgcaacgccttggaatagctcaggctgctttagatgaagagataaaatatgcaaaggaaagacaacagtttggaagaccaattggaaaatttcaaggcattcaatggtatatagctgatatggcaacgagaataaatgcttcaagatggcttgtatacaatgccgcttggagaaagcaggtaggtcttccgtacacaatggaagcagctatggcaaaattatatgcttccgaaacagcaatgtttgtaacgacaaaaacagttcagatatttggcggctatggctttacaaaagattatccagtggaaagatttatgagagatgcaaaaataacagaaatttatgaaggcacatcggaagtccagaaaatggttatttccggtaacctattgaaaatgtaaggagggcacacttatgaatatacttgttttgattaagcaggttccggatacgaacgaagttagaattgatcctgtaacaaagacacttatacgtgaaggtgttccaagcattattaatcctgatgacaaaaatgccttagaagaagctatcaggataagagaaaaagtcggcggaaaagtaactgtaatatcgatgggaccaacacaagcagaagtagcattacgtgaggctcttgctatgggtgctgatgaagcatatcttttgacagatagagcatttgcaggcgcagacacttatgctactgcaaaagcactatcaaaagcaattgaaaagtttcagtacgacattgtattttgcggcagacaggcgatagatggcgatactgcacaagttggaccacaaattgcagaacaattagacatacctcaggtaacatatgtaaggaaggtagaaatagaaggagacaaactcatagttgaaagagctcttgaagatggatatgaaatcattgaagttaagacgccagtcttattgacagctattaaagaacttaatgtgcctaggtatccttcaataaaaggaatattcaatgcttacaataaaaaagaagttaaaatcttaactgcagatgatttagaagttgataaaaatgaacttggactaaaaggttcaccaactaaggttgtagcaacgtcaactccaaatactgaaagagctggagaaatatttactggcaatataaaagaagctgttcagaatctagttgaaagattaaacagcagacatgtgatataggaggcgcgatggatgaacgattataaagatatatggatttttgcagaacaaagaaatggaaaacttatgaatgttgccatcgagatattaggtgaggcaagaaagcttgcagacaagaaaggcgttaatgttggagctgttttaatagggcacaatgtagaaaatctttcaaaagatcttataagttttggcgctgacatagtttatgttgtagacaatcctcttctttcaaattacacaaccgagggctatgcaaaggcaatatctgagcttgcaaaagagtataaaccagaagtcatactctatggagcaacttttataggtcgtgatcttgcaccgaggatagcatctagacttatgacagggcttactgcagattgtacagggctagatattgacgaaaatggactgctactgcaaacaaggccggcatttggaggaaacttaatggcaacaataaaatgtccagacaaaagaccacagatgtcgacagtaaggcctggagtcataaaagagctattagagatgatacaagagatgggaaaagtaattaaatttgatgctgatattaatgaaagtgatataagaactaagatattgagcatagcaaaagaagcaaaaaatgtagtgaaccttgaagaagctgacattatcgtttcaggcggacgtggaattggaggacctgatggatttaacatcataaaagaacttgcagacgtacggggaggcgtggttggcgcatctcgtgcaacagttgatgctgggtggataacatcagaccatcaagttggccagacaggtaagacagttagaccaaagctgtacattgcttgcggaattagtggtgcaatacagcatttagcaggtatgagcaactcaggtactattgtcgcaataaataaaaatcctgacgcgccgattttcaaatttgctgattacggcatagtcggggatttattcaaagtaataccagtattaatagaggaaatcaaaaagttaaaataaaggagctgtacatttgacagttgaccttaataaaaataaaaaggagtgtcccgggatgcaaaagatttgtgtaataggtgctggaacaatgggctcaggcatcgctcaagtatttgcacaaaatggctttgaagtaattttacgcgatattgatatgaagttcgtagaaaaaggatttggcacaattgaaaaaatttacaaagaaatgttgacaaagggaaaattacagcagatgagaaaacgaattttaagcagaatcagaggtacaacaaatttggaagacgcaaaagaagcagattttgtagttgaagcggctatagaaaatatggatctcaagaaacaaatattcaaagagctagatgaaatatgcaaaatggaaacaatccttgcgtcaaatacatcatcactatccataacagaaatagcaagtgcgacaaaaagacctgagaaagtcataggaatgcatttcttcaacccagttccagtaatgaaacttgttgaagtcataaaaggattaaagacatcagagcaaacatttaatgtcgtcagagaattggctttaaaagtagacaaaacacctatagaggtcaaagaagcacctggatttgttgtaaataggattttaatcccaatgattaatgaagcaattggaatacttgcagtggtgttggcaactgacaagagcatagatgaagctatgaaacttggtgcaaatcatccaataggacctttggcattgtctagtttgataggcaatgacgtcgttcttgctataatgaatgtgctttatgaagagtacggcgattcgaaatacagaccacatccacttctaaaaaaagtggtaagaggcggattgctgggtagaaaaactggcaaaggtttctttgaatacaaaattaatcttttaaggaggagaatatcatgaaagaagtagttattgcaagtggtgtaaggactgctgtcgggaaatttggtggcacgcttctaaatgtacctgcagtagatttaggtgctgtgaataataaaagaagcataaaaagagccaatgtgaaacctgaagatgttagtgaagtgataatgggaaatgtattgcaggcaggtcttgggcagaaccccgcaagacaagctgaaataaaagcgggcataccagtagaagttccggctatgactgtaaacatggtatgtggatcaggtcttagagctgtgacacttgctgctcaggcagttatgcttggtgatgctgacattgttgtagccggtggaatggaaaatatgtcaagagcaccatatatattaaatgatgctcgctttgggtacaggatgaacaatggccagcttgtagatgaaatggtatatgatggtttaacagatgtttttaaccaatatcacatgggaatcactgccgaaaatcttgctgaaaaatacggcatatcaagagaagagcaggatgaatttgcatatagaagccaaaaattagcgtcagaagcgatatcatcaggaagatttgaggatgagatagttcctgtgattgtgccgcagaaaaaaggtgaaccgatagaatttaaagttgatgaacatgtgagacctaatacgacaattgaagcacttgcaaaattaaaaccagcattccaaaaagatggaactgtaactgctggaaatgcatcaggaattaacgatgcagctgcagcagtagttgtgatgtcaaaagaaaaggcatgtgaacttggaataaagaccattgcaacgattaaatcatttggttatgcaggtgttgaccccagcatcacgggaattggtccagtatatgctacgagaaaggcattagaaaaagctaatctaactgtagatgatttagatttaattgaagcaaatgaagcatttgcagcacaatcactggctgttgcaaaagaattaaaatttaatatggacagagtgaatgtaaatggtggcgcaattgcgataggtcatccaatcggcgccagcggatgtagaattctagtgacgcttttatatgagatgcagaagaggaattcgcatactggacttgcaacattgtgcatcggcggaggaatgggaatagcaatggttgtcgaaagatag

(19) United States
(12) Patent Application Publication
(10) Pub. No.: US 2011/0097775 A1
(43) Pub. Date: Apr. 28, 2011

(54) PRODUCTION OF BUTANOL

(75) Inventors: Edward Green, Marlow (GB); Elizabeth Jenkinson, Oxford (GB); Mathew Crow, Oxfordshire (GB)

(73) Assignee: GREEN BIOLOGICS LIMITED, Oxfordshire (GB)

(21) Appl. No.: 12/936,020

(22) PCT Filed: Apr. 3, 2009
(86) PCT No.: PCT/GB09/00900

§ 371 Date: Dec. 17, 2010

(30) Foreign Application Priority Data
Apr. 3, 2008 (GB) 0806093.1

(51) Int. Cl. C12P 7/16; C12N 15/74; C12N 15/75; C12N 1/21

(52) U.S. Cl. 435/160; 435/471; 435/252.3

(57) ABSTRACT

The present invention relates to methods for making recombinant thermophilic bacteria of the family Bacillaceae. In particular, recombinant thermophilic Bacillaceae are provided which have been engineered to produce butanol and/or butyrate. Preferably, heterologous nucleic acid molecules encoding one or more butanol or butyrate biosynthetic pathway enzymes are introduced into a thermophilic Bacillaceae in order to produce a recombinant thermophilic Bacillaceae which is capable of producing butanol. The Bacillaceae is preferably of the genus Geobacillus or Ureibacillus. The invention also relates to a method of producing butanol using the Bacillaceae of the invention.

The present invention relates to methods for making recombinant thermophilic bacteria of the family Bacillaceae. In particular, recombinant thermophilic Bacillaceae are provided which have been engineered to produce butanol and/or butyrate. Preferably, heterologous nucleic acid molecules encoding one or more butanol and/or butyrate biosynthetic pathway enzymes are introduced into a thermophilic Bacillaceae in order to produce a recombinant thermophilic Bacillaceae which is capable of producing butanol and/or butyrate. The Bacillaceae is preferably of the genus Geobacillus or Ureibacillus.

The fermentation of carbohydrates to organic solvents such as acetone, ethanol and butanol has been known for nearly 100 years. U.S. Pat. No. 1,315,585 (1919) describes such a process using bacterial “found in soil and cereals, e.g., maize, rice, flax, etc.”. Such fermentation has traditionally been carried out using Clostridium acetobutylicum. Clostridium acetobutylicum is a gram-positive, sporulating, obligate anaerobe capable of naturally producing acetone, butanol, and ethanol (ABE).

Up until the 1950's, commercial production of ABE was economically competitive with petrochemical production, but with the advent of low cost crude oil and alternative cheaper production methods for ABE, methods involving Clostridium acetobutylicum became less attractive. With the current increased interest in non-petrochemical means of producing ABE and an increased global awareness of environmental issues, bacterial production of ABE has recently become more popular. Numerous different processes have or are being explored in order to increase yield of the ABE solvent, using naturally-occurring Clostridium or bacteria of other genera.

Attempts have also been made to insert the genes coding for butanol synthesis into organisms which do not naturally produce butanol. For example, WO2007/041269 discloses the production of butanol in genetically engineered mesophilic micro-organisms such as E. coli, B. subtilis and S. cerevisiae. The latter organisms were transformed with six genes involved in the synthesis of 1-butanol.

The current inventors have recognised for the first time, however, that particular advantages may be obtained from the production of butanol in thermophilic organisms that have been modified to express one or more butanol biosynthetic genes.

Thermophilic bacteria are generally isolated from high temperature environments (e.g. 55-70° C.). Such organisms have previously been exploited for the production of thermostable enzymes such as proteases, amylases and, of particular importance to molecular biology, DNA polymerases. However, thermophiles have not previously been used for industrial processes such as the production of solvents.

The use of high temperature technology has many advantages over mesophilic processes. The increased metabolic activity of thermophilic bacteria results in more efficient product formation and, furthermore, the use of higher temperatures eliminates the problem of contamination by mesophilic organisms. Thermophiles are generally able to utilize a wide-range of substrates derived from waste feedstocks, allowing the development of techniques for the production of second-generation biofuels that do not rely on valuable crop biomass.

Despite these advantages, developing thermophilic strains that are able to produce butanol is not a straightforward process. Almost all microbes that naturally produce butanol are mesophilic. The enzymes from these strains are unlikely to be functional in a thermophilic host primarily due to thermal instability. In addition, the tolerance of many thermophilic species to butanol is very low. WO2008/137402 discloses a method of producing butanol in mesophilic organisms in which the growth temperature is lowered to reduce the toxicity of the solvent. It has been suggested that the toxicity of butanol is due to interactions within components of cell membranes (Vollherbst-Schneck et al, Appl and Environ Micro, 47(1):193-194, (1984)). This effect may be exacerbated at higher temperatures with the increase in membrane fluidity.

Thermoanaerobacterium thermosaccharolyticum and Clostridium sp. AH1 are both natural thermophilic butanol producers. Clostridium sp. AH1 disclosed in U.S. Pat. No. 4,443,542 (1984) was found to produce limited amounts of butanol when grown on cellulose. Similarly a mutant of T. thermosaccharolyticum has also been shown to produce small amounts of butanol.

However, in both cases it is only a fraction of the butanol which could be extracted from C. acetobutylicum fermentations and T. thermosaccharolyticum requires the addition of extra butyrate to the growth media. T. thermosaccharolyticum has a low butanol tolerance and is a strict anaerobe which makes strain handling and genetic manipulation difficult. For these reasons we have looked to other sources for thermophilic strains that will be good hosts for the butanol synthesis pathway.

In one aspect, the invention provides a method for producing a recombinant Bacillaceae comprising the step of introducing one or more nucleic acid molecules which encode one or more butanol biosynthetic pathway polypeptides into a thermophilic Bacillaceae.

Preferably, the one or more butanol biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol.

Preferably, the introduction of the one or more nucleic acid molecules which encode one or more butanol biosynthetic pathway polypeptides results in a recombinant Bacillaceae which is capable of producing butanol.

In some embodiments, the (starting) thermophilic Bacillaceae is a non-butanol producing Bacillaceae.

In yet other embodiments, the (starting) thermophilic Bacillaceae is a non-butanol producing Bacillaceae and the introduction of the one or more nucleic acid molecules which encode one or more butanol biosynthetic pathway polypeptides results in a recombinant Bacillaceae which is capable of producing butanol.

In a further aspect, the invention provides a method for producing a recombinant Bacillaceae comprising the step of introducing one or more nucleic acid molecules which encode one or more butyrate biosynthetic pathway polypeptides into a thermophilic Bacillaceae.

Preferably, the one or more butyrate biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V′. butyryl-CoA to butyryl phosphate
VI′. butyryl phosphate to butyrate.

Preferably, the introduction of the one or more nucleic acid molecules which encode one or more butyrate biosynthetic pathway polypeptides results in a recombinant Bacillaceae which is capable of producing butyrate.

In some embodiments, the (starting) thermophilic Bacillaceae is a non-butyrate producing Bacillaceae.

In yet other embodiments, the (starting) thermophilic Bacillaceae is a non-butyrate producing Bacillaceae and the introduction of the one or more nucleic acid molecules which encode one or more butyrate biosynthetic pathway polypeptides results in a recombinant Bacillaceae which is capable of producing butyrate.

Optionally, the methods include the additional step of producing a master cell bank and/or working cell bank of the recombinant Bacillaceae; and growing a recombinant Bacillaceae from the master and/or working cell banks.

Alternatively, the methods include the additional step of storing aliquots of the recombinant Bacillaceae at a temperature of 5° C. or lower (preferably at a temperature lower than −15° C.); and subsequently growing the recombinant Bacillaceae on a solid media or in a liquid media.

A further aspect of the invention provides a method for producing a recombinant thermophilic Bacillaceae which is capable of producing butanol, comprising the steps:

(a) selecting a population of thermophilic Bacillaceae
(i) which do not produce butanol,
(ii) which are butanol-tolerant, and
(iii) which have one or more genes encoding butanol biosynthetic pathway polypeptides endogenously present in their genome,
   wherein the butanol biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol
   and
   (b) introducing into a thermophilic Bacillaceae from the selected population nucleic acid molecules coding for one or more, and preferably all, of those enzymes I-VI which were not endogenously present in the genome of the thermophilic Bacillaceae, in order to produce a recombinant thermophilic Bacillaceae which is capable of producing butanol.

Optionally, the method includes the additional step of (c) producing a master cell bank and/or working cell bank of the recombinant Bacillaceae, and growing a culture from the master and/or working cell banks.

Alternatively, the method includes the additional step of storing aliquots of the recombinant Bacillaceae at a temperature of 5° C. or lower (preferably at a temperature lower than −15° C.); and subsequently growing the recombinant Bacillaceae on a solid media or in a liquid media.

A further aspect of the invention provides a method for producing a recombinant thermophilic Bacillaceae, preferably a thermophilic Geobacillus or Ureibacillus, which is capable of producing butanol, comprising the steps:

(a) selecting a population of thermophilic Bacillaceae, preferably a thermophilic Geobacillus or Ureibacillus, which are butanol-tolerant, and
(b) introducing into a thermophilic Bacillaceae from the selected population nucleic acid molecules coding for the enzymes Crotonase (4.2.1.17), Butyryl CoA dehydrogenase (1.3.99.2), EtfB, EtfA, 3-hydroxybutyryl CoA dehydrogenase (1.1.1.35) and Acetyl CoA acetyltransferase (2.3.1.9), preferably wherein the aforementioned nucleic acid molecules are present in a single operon,
in order to produce a recombinant thermophilic Bacillaceae, preferably a thermophilic Geobacillus or Ureibacillus, which is capable of producing butanol.

Preferably, one or more or all of the abovementioned nucleic acid molecules are from Thermoanaerobacterium thermosaccharolyticum. Preferably, the operon is one having the sequence of SEQ ID NO: 36 or a variant or derivative thereof, encoding the above-mentioned enzymes. Optionally, nucleic acid molecules coding for enzymes 1.2.1.10 and/or 1.3.99.2 (and its associated cofactors EtfAB)/1.3.1.44 are also introduced into the thermophilic Bacillaceae. In some preferred embodiments, the thermophilic Bacillaceae is Geobacillus thermoglucosidasius.

A further aspect of the invention provides a method for producing a recombinant thermophilic Bacillaceae which is capable of producing butyrate, comprising the steps:

(a) selecting a population of thermophilic Bacillaceae
(i) which do not produce butyrate,
(ii) which are butyric acid tolerant, and
(iii) which have one or more genes encoding butyrate biosynthetic pathway polypeptides endogenously present in their genome,
   wherein the butyrate biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V′. butyryl-CoA to butyryl phosphate
VI′. butyryl phosphate to butyrate
   and
   (b) introducing into a thermophilic Bacillaceae from the selected population nucleic acid molecules coding for one or more, and preferably all, of those enzymes I-VI′ which were not endogenously present in the genome of the thermophilic Bacillaceae, in order to produce a recombinant thermophilic Bacillaceae which is capable of producing butyrate.

Optionally, the method includes the additional step of (c) producing a master cell bank and/or working cell bank of the recombinant Bacillaceae, and growing a culture from the master and/or working cell banks.

Alternatively, the method includes the additional step of storing aliquots of the recombinant Bacillaceae at a temperature of 5° C. or lower (preferably at a temperature lower than −15° C.); and subsequently growing the recombinant Bacillaceae on a solid media or in a liquid media.

In some preferred embodiments, one or more nucleic acid molecules which encode 1, 2, 3, 4, 5 or 6 of enzymes I-VI are introduced into the thermophilic Bacillaceae.

In other preferred embodiments, one or more nucleic acid molecules which encode enzymes I-II, I-III, I-IV, I-V or I-VI are introduced into the thermophilic Bacillaceae.

In other preferred embodiments, one or more nucleic acid molecules which encode enzymes II-III, II-IV, II-V or II-VI are introduced into the thermophilic Bacillaceae.

In other preferred embodiments, one or more nucleic acid molecules which encode enzymes III-IV, III-V or III-VI are introduced into the thermophilic Bacillaceae.

In other preferred embodiments, one or more nucleic acid molecules which encode enzymes IV-V or IV-VI are introduced into the thermophilic Bacillaceae.

In other preferred embodiments, one or more nucleic acid molecules which encode enzymes V-VI are introduced into the thermophilic Bacillaceae.

In other preferred embodiments, one or more nucleic acid molecules which encode enzyme VI are introduced into the thermophilic Bacillaceae.

In some embodiments, the thermophilic Bacillaceae is one which has endogenous genes coding for 1, 2, 3, 4, 5 or 6 of enzymes I-VI.

Preferably, the Bacillaceae have endogenous genes that encode enzymes I-II, I-III, I-IV, I-V or I-VI.

In other preferred embodiments, the Bacillaceae have endogenous genes that encode enzymes II-III, II-IV, II-V or II-VI.

In other preferred embodiments, the Bacillaceae have endogenous genes that encode enzymes III-IV, III-V or III-VI.

In other preferred embodiments, the Bacillaceae have endogenous genes that encode enzymes IV-V or IV-VI.

In other preferred embodiments, the Bacillaceae have endogenous genes that encode enzymes V-VI.

In other preferred embodiments, the Bacillaceae have an endogenous gene that encodes enzyme VI.

Preferably, the thermophilic Bacillaceae is one which has endogenous genes coding for 1, 2, 3, 4, or 5 of enzymes I-VI and the one or more nucleic acid molecules which are introduced into the thermophilic Bacillaceae encode one or more, and preferably all, of those enzymes I-VI which are not endogenously present in the thermophilic Bacillaceae.

In some embodiments of the invention, a single enzyme may be used to catalyse two of reactions I-VI, in place of two separate enzymes. Preferably, a single enzyme is used to catalyse reactions V+VI, e.g. acetaldehyde dehydrogenase (EC 1.2.1.10).

The embodiments and preferences and referred to herein with regard to enzymes V and VI apply also to enzymes V′ and VI′, mutatis mutandis.

In some particularly preferred embodiments, the Bacillaceae have endogenous genes which encode enzymes I, II and III and nucleic acid molecules coding for enzymes which catalyse reactions IV, V and VI are introduced into the thermophilic Bacillaceae. Most preferably, one enzyme (e.g. acetaldehyde dehydrogenase, EC 1.2.1.10) is used to catalyse reactions V and VI.

In other particularly preferred embodiments, nucleic acid molecules coding for enzymes EC 1.2.1.10 and EC 1.3.99.2 are introduced (preferably in a single operon) into a thermophilic Bacillaceae (preferably a Geobacillus) which has endogenous genes coding for enzymes which are capable of catalysing reactions I-III.

The person skilled in the art will understand that the enzyme EC 1.3.99.2 (butyryl-CoA-dehydrogenase) requires the presence of the electron transfer cofactors EtfA and EtfB, or, equivalents, in order to function. Consequently, in embodiments of the invention where nucleic acid coding for EC 1.3.99.2 is used, and electron transfer cofactors EtfA and EtfB or their equivalents are not present in the Bacillaceae in question, nucleic acid molecules coding for EtfA and EtfB, or their equivalents, are also introduced into the Bacillaceae in question.

In some embodiments of the invention, EC 1.3.1.44 may be used instead of EC 1.3.99.2.

In a further aspect, the invention provides a method for producing a recombinant Bacillaceae

comprising the step of introducing one or more nucleic acid molecules which encode one or more butanol and/or butyrate biosynthetic pathway polypeptides into a thermophilic Bacillaceae.

Preferably, the one or more butanol and/or butyrate biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol
VII. butyryl-CoA to butyryl phosphate
VIII. butyryl phosphate to butyrate

Preferably, the introduction of the one or more nucleic acid molecules which encode one or more butanol and/or butyrate biosynthetic pathway polypeptides results in a recombinant Bacillaceae which is capable of producing butanol and butyrate.

In some embodiments, the (starting) thermophilic Bacillaceae is a non-butanol producing Bacillaceae.

In other embodiments, the (starting) thermophilic Bacillaceae is a non-butyrate producing Bacillaceae.

In yet other embodiments, the (starting) thermophilic, Bacillaceae is a non-butanol producing Bacillaceae and the introduction of the one or more nucleic acid molecules which encode one or more butanol biosynthetic pathway polypeptides results in a recombinant Bacillaceae which is capable of producing butanol and butyrate.

Optionally, the method includes the additional step of Producing a master cell bank and/or working cell bank of the recombinant Bacillaceae; and growing a recombinant Bacillaceae from the master and/or working cell banks.

Alternatively, the method includes the additional step of storing aliquots of the recombinant Bacillaceae at a temperature of 5° C. or lower (preferably at a temperature lower than −15° C.); and subsequently growing the recombinant Bacillaceae on a solid media or in a liquid media.

A further aspect of the invention provides a method for producing a recombinant thermophilic Bacillaceae which is capable of producing butanol and butyrate, comprising the steps:

(a) selecting a population of thermophilic Bacillaceae
(i) which do not produce butanol or butyrate,
(ii) which are butanol-tolerant and butyric acid tolerant, and
(iii) which have one or more genes encoding butanol and/or butyrate biosynthetic pathway polypeptides endogenously present in their genome,
   wherein the butanol and/or butyrate biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol
VII. butyryl-CoA to butyryl phosphate
VIII. butyryl phosphate to butyrate
   and
   (b) introducing into a thermophilic Bacillaceae from the selected population nucleic acid molecules coding for one or more, and preferably all, of those enzymes I-VIII which were not endogenously present in the genome of the thermophilic Bacillaceae, in order to produce a recombinant thermophilic Bacillaceae which is capable of producing butanol and butyrate.

Optionally, the method includes the additional step of (c) producing a master cell bank and/or working cell bank of the recombinant Bacillaceae, and growing a culture from the master and/or working cell banks.

Alternatively, the method includes the additional step of storing aliquots of the recombinant Bacillaceae at a temperature of 5° C. or lower (preferably at a temperature lower than −15° C.); and subsequently growing the recombinant Bacillaceae on a solid media or in a liquid media.

In some preferred embodiments, one or more nucleic acid molecules which encode 1, 2, 3, 4, 5, 6, 7 or 8 of enzymes I-VIII are introduced into the thermophilic Bacillaceae.

In other preferred embodiments, one or more nucleic acid molecules which encode enzymes I-II, I-III, I-IV, I-V, I-VI, I-VII or I-VIII are introduced into the thermophilic Bacillaceae.

In other preferred embodiments, one or more nucleic acid molecules which encode enzymes II-III, II-IV, II-V, II-VI, II-VII or II-VIII are introduced into the thermophilic Bacillaceae.

In other preferred embodiments, one or more nucleic acid molecules which encode enzymes III-IV, III-V, III-VI, III-VII or III-VIII are introduced into the thermophilic Bacillaceae.

In other preferred embodiments, one or more nucleic acid molecules which encode enzymes IV-V, IV-VI, IV-VII or IV-VIII are introduced into the thermophilic Bacillaceae.

In other preferred embodiments, one or more nucleic acid molecules which encode enzymes V-VI, V-VII or V-VIII are introduced into the thermophilic Bacillaceae.

In other preferred embodiments, one or more nucleic acid molecules which encode enzymes VI-VII or VI-VIII are introduced into the thermophilic Bacillaceae.

In other preferred embodiments, one or more nucleic acid molecules which encode enzymes VII-VIII are introduced into the thermophilic Bacillaceae.

In other preferred embodiments, one or more nucleic acid molecules which encode enzyme VIII are introduced into the thermophilic Bacillaceae.

In some embodiments, the thermophilic Bacillaceae is one which has endogenous genes coding for 1, 2, 3, 4, 5, 6, 7 or 8 of enzymes I-VIII.

Preferably, the Bacillaceae have endogenous genes that encode enzymes I-II, I-III, I-IV, I-V, I-VI, I-VII or I-VIII.

In other preferred embodiments, the Bacillaceae have endogenous genes that encode enzymes II-III, II-Iv, II-V, II-VI, II-VII or II-VIII.

In other preferred embodiments, the Bacillaceae have endogenous genes that encode enzymes III-IV, III-V, III-VI, III-VII or III-VIII.

In other preferred embodiments, the Bacillaceae have endogenous genes that encode enzymes IV-V, IV-VI, IV-VII or IV-VIII.

In other preferred embodiments, the Bacillaceae have endogenous genes that encode enzymes V-VI, V-VII or V-VIII.

In other preferred embodiments, the Bacillaceae have an endogenous gene that encode enzymes VI-VII or VI-VIII.

In other preferred embodiments, the Bacillaceae have an endogenous gene that encode enzymes VII-VIII.

In other preferred embodiments, the Bacillaceae have an endogenous gene that encodes enzyme VIII.

Preferably, the thermophilic Bacillaceae is one which has endogenous genes coding for 1, 2, 3, 4, 5, 6, or 7 of enzymes I-VIII and the one or more nucleic acid molecules which are introduced into the thermophilic Bacillaceae encode one or more, and preferably all, of those enzymes I-VIII which are not endogenously present in the thermophilic Bacillaceae.

In some embodiments of the invention, one or more nucleic acid molecules which encode an enzyme which catalyses the following reaction IX are introduced into the thermophilic Bacillaceae in place of one or more nucleic acid molecules which encode an enzyme which catalyses reaction III:
IX (3S)-3-hydroxyacyl-CoA to trans-2(or 3)-enoyl-CoA.

In other embodiments of the invention, one or more nucleic acid molecules which encode an enzyme which catalyses the following reaction X are introduced into the thermophilic Bacillaceae in place of one or more nucleic acid molecules which encode an enzyme which catalyses reaction IV:
X acyl-CoA to trans-didehydroacyl-CoA.

The invention also relates to a recombinant Geobacillus or Ureibacillus which comprises one or more heterologous nucleic acid molecules which encode one or more butanol biosynthetic pathway enzymes selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol

Preferably, the Geobacillus or Ureibacillus is one which is capable of producing butanol.

The invention also relates to a recombinant Geobacillus or Ureibacillus which comprises one or more heterologous nucleic acid molecules which encode one or more butyrate biosynthetic pathway enzymes selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V′. butyryl-CoA to butyryl phosphate
VI′. butyryl phosphate to butyrate

Preferably, the Geobacillus or Ureibacillus is one which is capable of producing butyrate.

In some preferred embodiments, the recombinant Geobacillus or Ureibacillus comprises one or more heterologous nucleic acid molecules which encode 1, 2, 3, 4, 5 or 6 of enzymes I-VI.

In other preferred embodiments, the recombinant Geobacillus or Ureibacillus comprises one or more heterologous nucleic acid molecules which encode enzymes I-II, I-III, I-IV, I-V or I-VI.

In other preferred embodiments, the recombinant Geobacillus or Ureibacillus comprises one or more heterologous nucleic acid molecules which encode enzymes II-III, II-IV, II-V or II-VI.

In other preferred embodiments, the recombinant Geobacillus or Ureibacillus comprises one or more heterologous nucleic acid molecules which encode enzymes III-IV, III-V or III-VI.

In other preferred embodiments, the recombinant Geobacillus or Ureibacillus comprises one or more heterologous nucleic acid molecules which encode enzymes IV-V or IV-VI.

In other preferred embodiments, the recombinant Geobacillus or Ureibacillus comprises one or more heterologous nucleic acid molecules which encode enzymes V-VI.

In other preferred embodiments, the recombinant Geobacillus or Ureibacillus comprises one or more heterologous nucleic acid molecules which encode enzyme VI.

In some embodiments, the Geobacillus or Ureibacillus is one which has endogenous genes coding for 1, 2, 3, 4, 5 or 6 of enzymes I-VI.

Preferably, the Geobacillus or Ureibacillus is one which has endogenous genes that encode enzymes I-II, I-III, I-IV, I-V or I-VI.

In other preferred embodiments, the Geobacillus or Ureibacillus is one which has endogenous genes that encode enzymes II-III, II-IV, II-V or II-VI.

In other preferred embodiments, the Geobacillus or Ureibacillus is one which has endogenous genes that encode enzymes III-IV, III-V or III-VI.

In other preferred embodiments, the Geobacillus or Ureibacillus is one which has endogenous genes that encode enzymes IV-V or IV-VI.

In other preferred embodiments, the Geobacillus or Ureibacillus is one which has endogenous genes that encode enzymes V-VI.

In other preferred embodiments, the Geobacillus or Ureibacillus is one which has an endogenous gene that encodes enzyme VI.

Preferably, the Geobacillus or Ureibacillus is one which has endogenous genes coding for 1, 2, 3, 4, or 5 of enzymes I-VI and one or more heterologous nucleic acid molecules which encode one or more, and preferably all, of those enzymes I-VI which were not endogenously present in the Geobacillus or Ureibacillus.

The embodiments and preferences and referred to herein with regard to enzymes V and VI apply also to enzymes V′ and VI′, mutatis mutandis.

In some particularly preferred embodiments, the Geobacillus or Ureibacillus have endogenous genes which encode enzymes I, II and III and one or more heterologous nucleic acid molecules coding for enzymes which catalyse reactions IV, V and VI. Most preferably, one enzyme (e.g. acetaldehyde dehydrogenase, EC 1.2.1.10) is used to catalyse reactions V and VI.

In other particularly preferred embodiments, heterologous nucleic acid molecules coding for enzymes EC 1.2.1.10 and EC 1.3.99.2 (and optionally EtfAB) or 1.3.1.44 are present (preferably in a single operon) in a Geobacillus or Ureibacillus which has endogenous genes coding for enzymes which are capable of catalysing reactions I-III. Preferably, the Geobacillus is G. thermoglucosidasius. In some embodiments, the nucleic acid molecule coding for enzyme EC 1.2.1.10 is from E. coli or B. thuringiensis. In some embodiments, the nucleic acid molecule coding for enzyme 1.3.99.2 is from Thermoanaerobacterium thermosaccharolyticum.

In a further preferred embodiment, heterologous nucleic acid molecules coding for enzymes Crotonase (4.2.1.17), Butyryl CoA dehydrogenase (1.3.99.2), EtfB, EtfA, 3-hydroxybutyryl CoA dehydrogenase (1.1.1.35) and Acetyl CoA acetyltransferase (2.3.1.9) are present in a single operon in a Geobacillus or Ureibacillus. Preferably, one or more or all of the aforementioned heterologous nucleic acid molecules are from Thermoanaerobacterium thermosaccharolyticum. Optionally, heterologous nucleic acid molecules coding for enzymes 1.2.1.10 and/or 1.3.99.2 (and its associated cofactors EtfAB)/1.3.1.44 are also present. Preferably, the operon is one having the sequence of SEQ ID NO: 36 or a derivative or variant thereof, encoding the same enzymes. The invention also relates to a nucleic acid molecule wherein the nucleotide sequence of the nucleic acid molecule consists of or comprises the sequence given in SEQ ID NO: 36, and a vector wherein the nucleotide sequence of the vector comprises a nucleotide sequence of SEQ ID NO: 36.

The invention also relates to a recombinant Geobacillus or Ureibacillus which comprises one or more heterologous nucleic acid molecules which encode one or more butanol and/or butyrate biosynthetic pathway enzymes selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol
VII. butyryl-CoA to butyryl phosphate
VIII. butyryl phosphate to butyrate

Preferably, the Geobacillus or Ureibacillus is one which is capable of producing butanol and butyrate.

In some preferred embodiments, the recombinant Geobacillus or Ureibacillus comprises one or more heterologous nucleic acid molecules which encode 1, 2, 3, 4, 5, 6, 7 or 8 of enzymes I-VIII.

In other preferred embodiments, the recombinant Geobacillus or Ureibacillus comprises one or more heterologous nucleic acid molecules which encode enzymes I-II, I-III, I-IV, I-V, I-VI, I-VII or I-VIII.

In other preferred embodiments, the recombinant Geobacillus or Ureibacillus comprises one or more heterologous nucleic acid molecules which encode enzymes II-III, II-IV, II-V, II-VI, II-VII or II-VIII.

In other preferred embodiments, the recombinant Geobacillus or Ureibacillus comprises one or more heterologous nucleic acid molecules which encode enzymes III-IV, III-V, III-VI, III-VII or III-VIII.

In other preferred embodiments, the recombinant Geobacillus or Ureibacillus comprises one or more heterologous nucleic acid molecules which encode enzymes IV-V, IV-VI, IV-VII or IV-VIII.

In other preferred embodiments, the recombinant Geobacillus or Ureibacillus comprises one or more heterologous nucleic acid molecules which encode enzymes V-VI, V-VII or V-VIII.

In other preferred embodiments, the recombinant Geobacillus or Ureibacillus comprises one or more heterologous nucleic acid molecules which encode enzymes VI-VII or VI-VIII.

In other preferred embodiments, the recombinant Geobacillus or Ureibacillus comprises one or more heterologous nucleic acid molecules which encode enzymes VII-VIII.

In other preferred embodiments, the recombinant Geobacillus or Ureibacillus comprises one or more heterologous nucleic acid molecules which encode enzyme VIII.

In some embodiments, the Geobacillus or Ureibacillus is one which has endogenous genes coding for 1, 2, 3, 4, 5, 6, 7 or 8 of enzymes I-VIII.

Preferably, the Geobacillus or Ureibacillus is one which has endogenous genes that encode enzymes I-II, I-III, I-IV, I-V, I-VI, I-VII or I-VIII.

In other preferred embodiments, the Geobacillus or Ureibacillus is one which has endogenous genes that encode enzymes II-III, II-IV, II-V, II-VI, II-VII or II-VIII.

In other preferred embodiments, the Geobacillus or Ureibacillus is one which has endogenous genes that encode enzymes III-IV, III-V, III-VI, III-VII or III-VIII.

In other preferred embodiments, the Geobacillus or Ureibacillus is one which has endogenous genes that encode enzymes IV-V, IV-VI, IV-VII or IV-VIII.

In other preferred embodiments, the Geobacillus or Ureibacillus is one which has endogenous genes that encode enzymes V-VI, V-VII or V-VIII.

In other preferred embodiments, the Geobacillus or Ureibacillus is one which has an endogenous gene that encode enzymes VI-VII or VI-VIII.

In other preferred embodiments, the Geobacillus or Ureibacillus is one which has an endogenous gene that encode enzymes VII-VIII.

In other preferred embodiments, the Geobacillus or Ureibacillus is one which has an endogenous gene that encode enzyme VIII.

Preferably, the Geobacillus or Ureibacillus is one which has endogenous genes coding for 1, 2, 3, 4, 5, 6 or 7 of enzymes I-VIII and one or more heterologous nucleic acid molecules which encode one or more, and preferably all, of those enzymes I-VIII which are not endogenously present in the Geobacillus or Ureibacillus.

Preferably, the recombinant Geobacillus or Ureibacillus comprises one or more antibiotic resistance genes. Examples of such genes include genes conferring resistance to ampicillin, erythromycin, neomycin/kanamycin, tetracyline, chloramphenicol, spectinomycin, bleomycin or puromycin.

In some embodiments, the recombinant Geobacillus or Ureibacillus comprises one or more genes conferring tolerance to one or more heavy metals, e.g. mercury.

In some embodiments, one or more of the nucleic acid molecules comprises a heterologous promoter operably linked to a nucleic acid molecule which codes for one or more butanol and/or butyrate biosynthetic pathway enzymes.

Suitable promoters include inducible promoters, such as those that are inducible with specific sugars or sugar analogues, e.g. arabinose (e.g. lac, ara), those inducible with tetracycline (e.g. tet), those inducible with IPTG (e.g. trp, tac, Pspac), those inducible with heat (e.g. hsp70), those inducible with anaerobic induction (e.g. nisA, pfl), P11, ldh, SV40 promoter, those inducible with xylose (e.g. Pxyl promoter), those inducible with osmotic shock, cell density (quorum sensing), antibiotics, or growth phase.

In some embodiments, the promoter is a constitutive promoter (e.g. secDF, β-Gal).

In other embodiments, the promoter is one from Clostridia, e.g. a promoter from the pta/ptb genes.

In other embodiments, the genome of the Geobacillus or Ureibacillus has an insertion in one or more genes, such as an insertion in the lactate dehydrogenase (ldh) gene and/or in the ethanol dehydrogenase gene, thus rendering those genes non-functional.

In yet other embodiments, the Geobacillus or Ureibacillus comprises one more plasmids, e.g. ‘bacillus plasmids’, plasmids for Gram positive bacteria derived from pUB110, pAYC36, pAYC37, pUBUC, pNW33N, pC194, pS194, pSA2100, pE194, pT127, pUB112, pC221, pC223, pAB124, or pBD.

In yet other embodiments, the Geobacillus or Ureibacillus comprises a fragment from a plasmid from an E. coli vectors, e.g. a fragment of pUC series plasmid, pGEM, pACYC184, pTOPO, pBR322, pFB series, pBluescript, p15A, F, RP4, RSF1010, ColE1.

In yet other embodiments, the Geobacillus or Ureibacillus comprises a non-native transposon insertion sequence, e.g. one from Tn 10, Tn 5, Tn 1545, Tn 916, or ISCb 1.

As used herein, the term “thermophilic” refers to bacterium which is capable of growing at a temperature of above 50° C., preferably above 55° C., more preferably above 60° C., and most preferably above 65° C. or above 70° C.

In other embodiments, the term “thermophilic” refers to bacterium which is capable of growing at a temperature of 40-75° C., 50-75° C., 60-75° C., 70-75° C., 55-70° C., 55-65° C. or 55-60° C.

In other embodiments, the term “thermophilic” refers to bacterium which is not capable of growing or only grows to a minimal extent below 25° C.

The thermophilic bacteria of the family Bacillaceae is preferably a thermophilic Bacillaceae of Anoxybacillus, Bacillus, Brevibacillus, Paenibacillus, Hydrogenophilus, Geobacillus orUreibacillus. More preferably, the bacteria is a Geobacillus or Ureibacillus.

Most preferably, the bacteria is a Geobacillus.

In some embodiments of the invention, the Bacillaceae is an aerobic Bacillaceae. In other embodiments, the Bacillaceae is an anaerobic Bacillaceae.

In yet other embodiments of the invention, the Bacillaceae is a facultative anaerobic Bacillaceae. In other embodiments, the Bacillaceae is not an obligate anaerobic Bacillaceae.

In some preferred embodiments, the Bacillaceae is a facultative anaerobic Geobacillus or Ureibacillus.

In some embodiments of the invention, the Bacillaceae is a Gram +ve Bacillaceae. In other embodiments, the Bacillaceae is a Gram −ve Bacillaceae.

Bacteria of the above genera may be characterised by standard methods known the art. Examples include “Bergey's Manual of Systematic Bacteriology” (Editor-in-chief: Garrity, George M., publ. Springer) and methods involving the analysis of 16S rRNA (e.g. Fox et al. (1980). “The phylogeny of prokaryotes”. Science 209:457-463

The Geobacillus is preferably Geobacillus thermoglucosidasius, Geobacillus kaustophilus, Geobacillus subterraneus, Geobacillus uzenensis, Geobacillus thermocatenulatus, Geobacillus thermodenitrificans, Geobacillus thermoleovorans, Geobacillus thermantarticus, Geobacillus pallidus, Geobacillus toebii, Geobacillus caldoxylosilyticus, Geobacillus tropicalis or Geobacillus stearothermophilus.

Most preferably, the Geobacillus is Geobacillus thermoglucosidasius, Geobacillus stearothermophilus or Geobacillus thermodenitrificans.

Examples of Geobacillus thermoglucosidasius include DSM 2542 (BGSC Accession No. 95A1), NCIMB 11955.

Examples of Geobacillus stearothermophilus include strain DSM 22 and NCA 1503.

Examples of Geobacillus thermodenitrificans include strain DSM 465.

In some embodiments of the invention, the Geobacillus strain defined in JP2005-261239 by deposit FERM P-19706 is specifically excluded.

In some embodiments of the invention, Geobacillus kaustophilus is specifically excluded.

Preferred strains of Ureibacillus include Ureibacillus thermosphaericus and Ureibacillus terrenus.

In some embodiments of the invention, Thermoanaerobacterium thermosaccharolyticum is specifically excluded.

In yet other embodiments of the invention, Clostridium sp. AH1 (FERM-P6093, ATCC 39045) is specifically excluded.

Preferably the bacteria is a butanol-tolerant Bacillaceae.

As used herein, the term “butanol-tolerant” refers to a bacterium which is tolerant of at least 0.5% weight/volume butanol, preferably at least 1.0% weight/volume butanol, more preferably at least 1.5% weight/volume butanol and most preferably at least 2.0% weight/volume butanol. Particularly preferred “butanol-tolerant” bacteria are those which are tolerant to at least 2.5% weight/volume butanol.

The same applies to bacteria which are butyrate tolerant, mutatis mutandis.

The recombinant Bacillaceae of the invention and used in the invention are preferably ones which are capable of producing 0.1 g butanol/g sugar, more preferably 0.2 g butanol/g sugar and most preferably 0.4 g butanol/g sugar.

In some embodiments of the invention, no butyrate or no additional butyrate is added to the growth medium.

As used herein the term “nucleic acid molecule” refers to a DNA or RNA molecule, which might be single- or double stranded. Preferably, the nucleic acid molecule is a DNA molecule, most preferably a double-stranded DNA molecule.

The nucleic acid molecule is preferably one which contains no introns. The nucleic acid molecule may, for example, be intron-less genomic DNA or cDNA.

The nucleic acid molecule is introduced in a manner such that the one or more butanol/butyrate biosynthetic pathway polypeptides are expressed in the Bacillaceae.

As used herein, the term “introduced” refers primarily to a nucleic acid molecule which is introduced into the Bacillaceae by any means. Examples of suitable means include transformation, transfection and electroporation.

As used herein, the term “heterologous” refers to a nucleic acid molecule which is not present in the naturally-occurring form of the Bacillaceae into which the nucleic acid is being introduced. In other words, a heterologous nucleic acid is one which is foreign to the Bacillaceae into which it is being introduced.

As used herein, the term “butanol biosynthetic pathway polypeptide” refers to an enzyme which is involved in the conversion of acetyl-CoA to butanol, in particular an enzyme which catalyses one or more of the following steps:
I acetyl-CoA to acetoacetyl-CoA
II acetoacetyl-CoA to 3-hydroxybutyryl-CoA
III 3-hydroxybutyryl-CoA to crotonyl-CoA
IV crotonyl-CoA to butyryl-CoA
V butyryl-CoA to butyraldehyde and
VI butyraldehyde to 1-butanol.

As used herein, the term “butyrate biosynthetic pathway polypeptide” refers to an enzyme which is involved in the conversion of acetyl-CoA to butyrate, in particular an enzyme which catalyses one or more of the following steps:
I acetyl-CoA to acetoacetyl-CoA
II acetoacetyl-CoA to 3-hydroxybutyryl-CoA
III 3-hydroxybutyryl-CoA to crotonyl-CoA
IV crotonyl-CoA to butyryl-CoA
V′. butyryl-CoA to butyryl phosphate
VI′. butyryl phosphate to butyrate

Examples of butanol and butyrate biosynthetic pathway polypeptides are given in Table 1 below:

TABLE 1

| | | |
|---|---|---|
| EC 2.3.1.9 | Acetyl-CoA | 2 acetyl-CoA = CoA + |
| | Acetyltransferase | acetoacetyl-CoA |
| EC 1.1.1.157/ | 3-hydroxybutyryl-CoA | (S)-3-hydroxybutanoyl-CoA + |
| EC 1.1.1.35 | dehydrogenase | NADP⁺ = 3-acetoacetyl-CoA + |
| | | NADPH + H⁺ |
| EC 4.2.1.17 | enoyl-CoA hydratase | (3S)-3-hydroxyacyl-CoA = |
| | | trans-2(or 3)-enoyl-CoA + H₂O |
| EC 4.2.1.55 | 3-hydroxybutyryl-CoA | (3R)-3-hydroxybutanoyl-CoA = |
| | dehydratase | crotonoyl-CoA + H₂O |
| EC 1.3.99.2 | butyryl-CoA | butanoyl-CoA + acceptor = 2- |
| | dehydrogenase | butenoyl-CoA + reduced |
| | | acceptor |
| EC 1.3.1.44 | trans-2-enoyl-CoA | acyl-CoA + NAD⁺ = trans- |
| | reductase (NAD⁺) | didehydroacyl-CoA + NADH + |
| | | H⁺ |
| EC 1.2.1.10 | acetaldehyde | acetaldehyde + CoA + NAD⁺ = |
| | dehydrogenase | acetyl-CoA + NADH + H⁺ |
| | (acetylating) | |
| EC 1.1.1.1 | alcohol dehydrogenase | an alcohol + NAD⁺ = an |
| | | aldehyde or ketone + NADH + |
| | | H⁺ |
| EC 2.3.1.19 | phosphate | butanoyl-CoA + phosphate = |
| | butyryltransferase | CoA + butanoyl phosphate |
| EC 2.7.2.7 | butyrate kinase | ATP + butanoate = ADP + |
| | | butanoyl phosphate |

The above reactions are as defined by the Nomenclature Committee of the International Union of Biochemistry and Molecular Biology (NC-IUBMB) (in consultation with the IUPAC-IUBMB Joint Commission on Biochemical Nomenclature (JCBN)) (http://www.chem.qmul.ac.uk/idiubmb/enzyme/).

Examples of suitable sources of the butanol and/or butyrate biosynthetic genes are given in the Examples herein and in WO2007/041269 (particularly pages 8-10 and 15-19), the contents of which are specifically incorporated herein by reference.

Preferably, the nucleic acid molecules encoding the butanol biosynthetic pathway polypeptide or butyrate biosynthetic pathway are obtained from a thermophilic micro-organism, preferably a thermophilic bacteria.

In other embodiments, the butanol biosynthetic pathway polypeptides or butyrate biosynthetic pathway polypeptides are thermostable polypeptides (of natural or synthetic origin). As used herein, the term “thermostable” means that the polypeptide has a half-life at 50° C. which is at least 90%, preferably at least 100%, of the half-life when measured under equivalent conditions at 37° C.

In some embodiments of the invention, therefore, the nucleic acid molecule encodes a thermostable form the butanol/butyrate biosynthetic pathway polypeptide.

In some embodiments of the invention, the nucleic acid molecule encodes a form the butanol/butyrate biosynthetic pathway polypeptide which is more thermostable than the corresponding endogenous polypeptide.

Various methods exist for making thermostable proteins. One approach subjects the gene sequence to random mutations by sub-optimal PCR conditions (e.g. non-proofreading enzymes or replacement of Mg²⁺ with Mn²⁺). A number of mutated gene copies may then be cloned into an appropriate vector and transformed into the target strains. Butanol/butyrate production at elevated temperatures may then be measured. Clones showing the greatest thermostability may be recovered and the gene sequenced to determine the position of the beneficial mutations. These proteins may be his-tagged to enable expression profiles to be determined. Proteins can also then be purified from the target organism, e.g. via a single step affinity chromatography batch binding method using Ni²⁺ or Co²⁺. The properties of the soluble protein may then be investigated further. If multiple increased-stability mutants are obtained, then a single clone may be developed containing all of the beneficial mutations. This may then transformed into the target organism as the final modified thermostable clone.

The nucleic acid molecules will generally comprise a promoter which is operable in the host cell, and which is operably linked to the 5′-end of the nucleic acid sequence which codes for the butanol/butyrate biosynthetic pathway enzyme.

The nucleic acid molecule will generally comprise a terminator which is operable in the host cell, and which is operably linked to the 3′-end of the nucleic acid sequence which codes for the butanol/butyrate biosynthetic pathway enzyme.

In order to obtain optimal expression of the gene encoding the butanol/butyrate biosynthetic pathway polypeptide, it is desirable to use codons which are preferred by the host organism.

For sequenced genomes, codon tables exist that allow the re-coding of the gene of interest.

For unsequenced organisms, the products of degenerate PCRs can be used to estimate the preferred codon usage. Highly conserved genes (e.g. based on the sequenced Geobacillus strains, e.g. kaustophilus, thermodenitrificans and stearothermophilus) may be amplified using degenerate primers. The resultant products may be sequenced and back-translated into amino acids in order to identify the most commonly used codons. Once identified, the gene may be re-coded and synthesised (e.g. by GenScript). The optimised gene may then be cloned into an appropriate vector and transformed into the host organism.

In some embodiments of the invention, therefore, the nucleic acid molecule(s) encoding the butanol/butyrate biosynthetic pathway polypeptide(s) is/are codon-optimised for the host Bacillaceae.

The butanol/butyrate biosynthetic pathway genes will not necessarily be optimal at the growth temperature of the target organism.

The deletion of the lactate dehydrogenase (LDH, EC 1.1.1.27) gene in the Bacillaceae may increase the production of butanol or butyrate by diverting pyruvate away from lactate production and into butanol/butyrate production, i.e. more pyruvate will be available to channel into acetyl-CoA and the subsequent steps on the butanol/butyrate biosynthetic pathway. The LDH gene may identified by homology and degenerate PCR. A truncated version of the gene may be made by PCR and cloned into an appropriate vector. Homologous recombination may be used to knock out the endogenous LDH gene, replacing it with an inactive version. Alternatively, siRNA may be used to knock out the LDH gene.

A further aspect therefore provides a recombinant Bacillaceae of the invention which does not contain a functional lactate dehydrogenase gene or which does not produce lactate dehydrogenase or which produces a lower level of lactate dehydrogenase compared to a corresponding non-modified Bacillaceae.

A further aspect therefore provides a modified Bacillaceae of the invention which does not produce lactate or only produces trace amounts of lactate or which produces a lower level of lactate compared to a corresponding non-modified Bacillaceae.

In a similar manner, the genes encoding enzymes that are specific to the production of ethanol may be removed or knocked out.

A further aspect therefore provides a modified Bacillaceae of the invention which does not contain a functional ethanol dehydrogenase gene or which does not produce ethanol dehydrogenase or which produces a lower level of ethanol dehydrogenase compared to a corresponding non-modified Bacillaceae.

A further aspect therefore provides a modified Bacillaceae of the invention which does not produce ethanol or only produces trace amounts of ethanol or which produces a lower level of ethanol compared to a corresponding non-modified Bacillaceae.

There are also other genes which utilise pyruvate which may usefully be knocked out in order to optimize butanol/butyrate production. Examples include acetolactate synthase (EC 2.2.1.6) and phosphotransacetylase (EC 2.3.1.8).

A further aspect therefore provides a modified Bacillaceae of the invention which does not contain a functional acetolactate synthase (EC 2.2.1.6) and/or phosphotransacetylase (EC 2.3.1.8) gene or which does not produce acetolactate synthase (EC 2.2.1.6) and/or phosphotransacetylase (EC 2.3.1.8) or which produces a lower level of acetolactate synthase (EC 2.2.1.6) and/or phosphotransacetylase (EC 2.3.1.8) compared to a corresponding non-modified Bacillaceae.

The person skilled in the art will be aware of the fact that genetically modifying the core metabolic pathways of Bacillaceae species may cause cells to become unstable. In particular, growth rates may be affected. One cause of this is an imbalance in co-factor ratios caused by knocking out some of the pathways, e.g. the NADH/NAD⁺ ratio is essential for ensuring each step in the pathway can be completed. Knock-on effects in the glycolysis pathway may also cause cells to become unstable. A method for regenerating NADH/NAD⁺ requires the introduction of NAD⁺-dependent formate dehydrogenase (FDH, EC 1.2.1.2). NAD+-dependent FDH from an appropriate organism may be codon-optimized for expression in the desired host. The gene may be cloned into a shuttle vector under a suitable promoter and either expressed from a plasmid or inserted into the host genome using homologous recombination.

The invention therefore provides a recombinant Bacillaceae of the invention which additionally comprises a heterologous nucleic acid which encodes a NAD⁺-dependent formate dehydrogenase, preferably one which is codon-optimised for the host organism.

Methods of transformation of genera within the Bacillaceae family are well known in the art.

A variety of methods are known for the transformation of Geobacillus strains. Examples of the transformation of G. stearothermophilus protoplasts and of the electroporation of G. stearothermophilus are given in the Bacillus Genetic Stock Center Catalog of Strains, Seventh Edition, Vol. 3: The Genus Geobacillus http://www.bgsc.org/Catalogs/Catpart3.pdf) (see also Narumi et al. 1993. “Construction of a New Shuttle Vector pSTE33 and Its Stabilities in Bacillus stearothermophilus, Bacillus subtilis, and Escherichia coli.” Biotechnology Letters 15(8):815-820; Narumi, et al. 1992. “A newly isolated Bacillus stearothermophilus K1041 and its transformation by electroporation. Biotechnol. Techniques” 6(83-86); and Wu, et al. 1989. “Protoplast Transformation of Bacillus stearothermophilus NUB36 By Plasmid DNA”. Journal of General Microbiology 135:1315-1324).

The two main techniques involve:

1) Washing the bacteria in a solution that will prevent the cells from lysing e.g sucrose and then mixing with the plasmid DNA. Subjecting this mix to an electric current causes the opening of small pores in the cell membrane which reseal during recovery growth. This method tends to be fairly high efficiency although the electroporation parameters need defining for each strain being transformed.
2) For Gram +ve bacteria the cells can be treated with lysozyme in order to produce protoplasts which are incubated with the DNA and PEG to induce the cells to take up the plasmids. Selection is carried out on plates in much the same way as for E. coli except antibiotic resistance, temperature and media all need to be optimised for these strains in order to promote efficient regeneration of the cell wall.

The nucleic acid molecules may be introduced into the Bacillaceae in any appropriate form, preferably in the form of a plasmid or vector. The nucleic acid molecules or parts thereof may become stably integrated into the Bacillaceae genome. In other embodiments, they are maintained as plasmids within the Bacillaceae.

The invention also relates to a process of producing butanol comprising culturing a recombinant Geobacillus or Ureibacillus of the invention together with an appropriate substrate.

The invention further provides a process of producing butyrate comprising culturing a recombinant Geobacillus or Ureibacillus of the invention together with an appropriate substrate.

Examples of suitable substrates include sugars such glucose, sucrose, fructose, xylose, galactose, mannose, mannitol, molasses (e.g. from the sugar industry), (e.g. from food waste or maize), black liquor (e.g. from the paper industry), hemicellulose, lignocellulose and cellulose (e.g. from plant material). Preferred carbon sources include glucose, sucrose, fructose, xylose, mannose, mannitol, cellulose and xylan.

The process of the invention may be operated in any suitable manner. For example, it may be operated as a batch process, fed-batch process or any form of continuous process.

Isolation of butanol and/or butyrate from the culture media may be carried out by any suitable means. Examples of include gas-stripping, pervaporation, distillation and solvent extraction.

### BRIEF DESCRIPTION OF THE FIGURES

FIGS. 1A-C show butanol tolerance of selected thermophilic strains.

FIG. 2 shows the outline of plasmids created for use in the genetic modification of thermophiles and sequences of N-terminal his tags.

FIG. 2A: The kanamycin cassette has been replaced with erythromycin, spectinomycin or tetracycline to create plasmids pPSM-E, pPSM-S and pPSM-T.

FIG. 2B: ‘Short’ His tag sequence. Oligos were annealed together and ligated downstream of pLdh in pPSM-K to create plasmid pPSM-pLdhHis.

FIG. 2C: Long' His tag sequence. Oligos for the multiple cloning site (MCS) were annealed and ligated downstream of the ‘short’ his tag to create pPSM-pLdhLHis.

FIG. 3 shows Western blot detection of N-terminally his-tagged proteins expressed in G. thermoglucosidasius.

FIG. 3A: Denaturing protein purification of transformants expressing Tats 1.3.99.2 and EtfA/B proteins. His tagged 1.3.99.2 at the expected size of 43 kDa (band highlighted) was detected using an anti-RGS primary antibody and anti-mouse secondary antibody.

FIG. 3B: Denaturing protein purification of transformants expressing Bt 1.2.1.10. His tagged 1.2.1.10 at the expected size of 39 kDa (band highlighted) was detected as for Tats 1.3.99.2.

FIG. 4: Butanol operon. ORFs are underlined; start codons are highlighted in gray (ATG); and putative ribosome binding sites are in bold italics. The ORFs are, in order: Crotonase (4.2.1.17), Butyryl CoA dehydrogenase (1.3.99.2), EtfB, EtfA, 3-hydroxybutyryl CoA dehydrogenase (1.1.1.35) and Acetyl CoA acetyltransferase (2.3.1.9). (The last 2 genes overlap).

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to the skilled person from the foregoing description. Such modifications are also intended to fall within the scope of the appended claims.

The disclosure of each reference set forth herein is incorporated herein by reference in its entirety.

### EXAMPLES

TABLE 1
Bacterial strains, genomic DNA and plasmid sources

| | Relevant | |
|---|---|---|
| | characteristics | Source/Reference |
| Strains | | |
| Haemophilus aegyptius | HaeIII methylase | HPA/NCTC |
| NCTC 8502 | | |
| Escherichia coli DH5α | Cloning strain | Invitrogen |
| Escherichia coli Novablue | Cloning strain | Merck (Novagen) |
| Escherichia coli DH10B | Cloning strain for | Invitrogen |
| | methylated plasmids | |
| Escherichia coli mHae | Methylation strain | This work |
| | harbouring HaeIII | |
| | methylase | |
| Geobacillus | Host strain | Elsworth |
| thermoglucosidasius TN-TK | | |
| Geobacillus kaustophilus | Donor of β-Gal gene | DSMZ |
| (DSM 7263) | and pGal promoter | |
| Bacillus thuringiensis | Donor of gene | N. Crickmore, |
| serovar israelensis | homologues | University of |
| | | Sussex |
| Thermoanaerobacterium | Donor of butanol | DSMZ |
| thermosaccharolyticum | operon | |
| (DSM 571) | | |
| Plasmids | | |
| pGem-T Easy | Holding vector | Promega |
| pSL1180 | Amp^{R}; Donor of MCS | GE Biosciences |
| pNW33N | Cam^{R} | Bacillus Stock |
| | | Centre |
| pNW-MCS | Cam^{R} | This work |
| pUBUC | Km^{R}/Amp^{R} | Elsworth |
| pUB-MCS | Km^{R}/Amp^{R} | This work |
| pPSM-K | Km^{R}/Amp^{R} | This work |
| pCL1920 | Spc^{R} | Lerner and |
| | | Inouye, 1990 |
| pLJ154 | Spc^{R}; cloned m.HaeIII | This work |

### Example 1

### Analysis of Bacterial Strains

### Butanol Tolerance

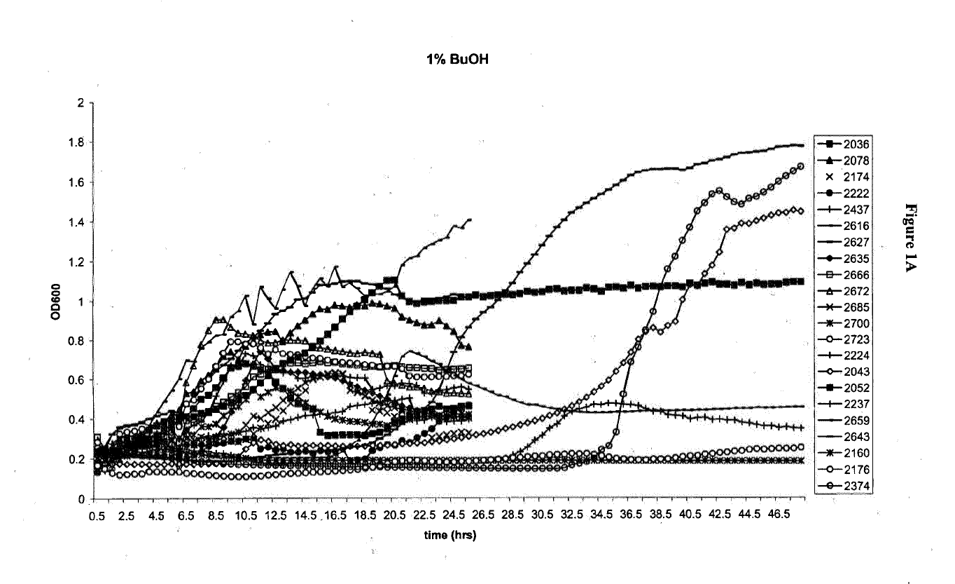

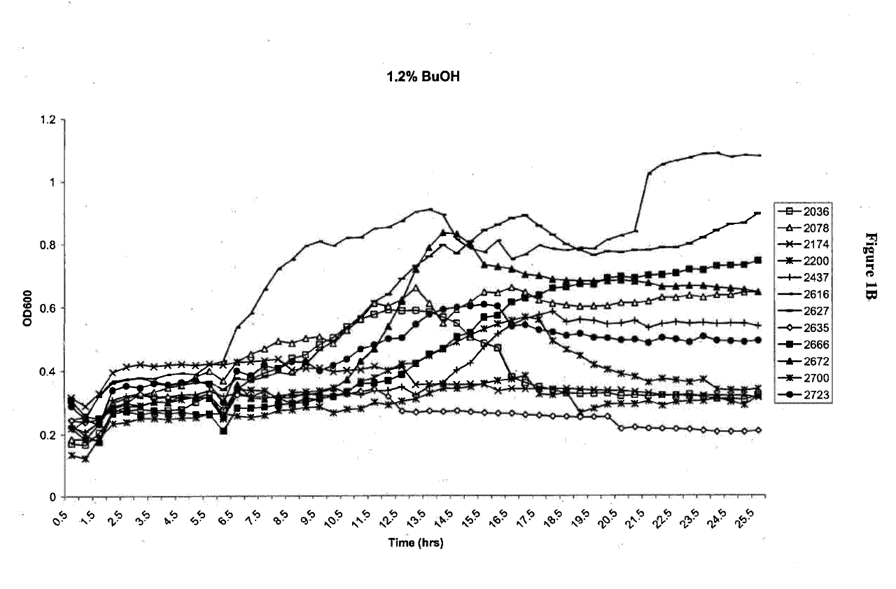

A number of thermophilic bacterial strains were tested for butanol tolerance. Overnight liquid cultures were incubated at 52-60° C. 40 μl was then used to inoculate 360 μl TSB or 2×YT containing 0-2% butanol in a 100 well bioscreen plate (Labsystems). Cultures were incubated at 52° C. for up to 48 hours without shaking and OD readings were taken at 600 nm every 30 mins (FIGS. 1A-C). A number of strains with tolerance to 1.2-1.4% butanol were identified for further characterisation.

### Butanol Pathway Investigations

Bacterial strains showing tolerance to butanol (0.5-2%) and/or butyric acid were investigated further to identify which genes are missing from the butanol production pathway (based on the Clostridium acetobutylicum pathway).

Proteins encoded by the following genes were identified as involved in butanol production:
EC 2.3.1.9 Acetyl-CoA acetyltransferase (THL)
EC 1.1.1.35/EC 1.1.1.1573-hydroxybutyryl-CoA dehydrogenase (HBD)
EC 4.2.1.55/EC 4.2.1.173-hydroxybutyryl-CoA dehydratase (CRT)
EC 1.3.99.2/EC 1.3.1.44 butyryl-CoA dehydrogenase (BCD)
EC 1.2.1.10 acetaldehyde dehydrogenase (acetylating)
EC 1.1.1.1 alcohol dehydrogenase

The following are involved in butyrate production:
EC 2.3.1.19 phosphate butyryltransferase
EC 2.7.2.7 butyrate kinase

Homologues of the above genes from a variety of micro-organisms were aligned (CLUSTALX) and conserved regions identified. Degenerate PCR primers were designed to these conserved regions (maximum degeneracy 4096×) and used to amplify gene fragments from gDNA prepared from organisms with good butanol tolerance.

gDNA Isolation

Cultures were pelleted, resuspended in 500 μl TE 8.0, and immediately frozen at −20° C. Frozen cells were thawed quickly at 37° C., vortexed and lysozyme added (final conc 1 ng/μl) before incubating the sample for 30 mins at 37° C. Cells were treated with SDS at 0.5%, and 50 μg proteinase K and incubated at 37° C. for 60 mins before NaCl was added to 900 mM and 75 μl CTAB/NaCl mix (10% CTAB, 0.7M NaCl stock solution) was added. Samples were mixed and incubated at 65° C. for at least 20 mins. Finally, gDNA was extracted using chloroform:IAA (24:1) followed by isopropanol precipitation. The resultant pellet was washed in 70% EtOH, air dried and resuspended in TE pH8.0. Samples were checked on a 1% TAE agarose gel.

### Degenerate PCR

PCR was carried out in 50 μl reactions using platinum supermix (Invitrogen) using ˜250 ng gDNA as the template and 50 pmols each degenerate primer (specific for the gene being amplified). Optimal amplification profiles were determined for each of the genes, modifying annealing temperatures and elongation times as required. Samples were analysed on 1-2% TAE agarose gels. The generation of a specific band of the predicted size was taken as positive for the presence of the gene within the tested genome. A sample were selected for confirmation by sequencing (GATC-Biotech), using the degenerate oligos as sequencing primers. The absence of any bands (compared with a positive control) or bands of incorrect sizes, were assumed to indicate that the gene was missing from the genome and hence in order for butanol production, such genes would need to be engineered into the target strain's genome.

### Example 2

### Preparation of Butanol Biosynthetic Pathway Genes

From the butanol production pathway, Geobacillus thermoglucosidasius was found to have homologues of genes 1.1.1.157 (1.1.1.35 not tested), both 4.2.1.55 and 4.2.1.17 and 1.1.1.1.

From the butyrate biosynthesis pathway, a homologue of 2.3.1.19 was identified in Geobacillus thermoglucosidasius.

In order to complete the main butanol biosynthetic pathway, at least genes 1.2.1.10 and 1.3.99.2 (and its associated co-factors, EtfA and EtfB) or 1.3.1.44 need to be added.

### Pathway Cloning

### Growth Conditions

E. coli which were used as plasmid holding and propagating strains were grown aerobically in either Luria-Bertani (LB) or 2× Yeast Tryptone (YT) medium supplemented with antibiotics as required: Ampicillin (100 μg/ml); Kanamycin (30 μg/ml); Chloramphenicol (25 μg/ml); Streptomycin sulphate (50 μg/ml); Spectinomycin (50 μg/ml).

Strains used for gDNA preparation were grown aerobically at their optimal growth temperatures in LB or TSB medium in the absence of antibiotic. Thermoanaerobacterium thermosaccharolyticum strains used for gDNA preparation were grown anaerobically at 60° C. in RCM.

1.2.1.10

A search of sequenced 1.2.1.10 genes using BRENDA (http://www.brenda-enzymes.info/) showed some of the genes classified as both Alcohol dehydrogenase (1.1.1.1) and acetaldehyde dehydrogenase (1.2.1.10), e.g. P0A9Q7 (E. coli) and Q3EZPO (B. thuringiensis) and others classified just as Acetaldehyde dehydrogenase e.g. Q5FLS6 (L. acidophilus). Potentially these genes will perform the function of both of the last two steps of the reaction:

Butyryl-CoA→Butyraldehyde→Butanol

The annotated B. thuringiensis gene is half the size of the E. coli variant which may indicate a functional difference as to whether both steps or just a single step is carried out.

### 1.3.99.2/EtfAB

1.3.99.2 homologues identified using BRENDA were classified as butyryl CoA dehydrogenases and perform a single reaction within the pathway:

Crotonoyl-CoA→Butyryl-CoA

They require 2 cofactor proteins in order to function, Electron Transfer Flavoproteins (Etf) A and B.

TABLE 2
Genes chosen for cloning and expression in selected thermophiles

| Gene | Ref. No. | Donor strain | Details |
|---|---|---|---|
| 1.2.1.10/ | Q3EZP0 | B. thuringiensis (Bt) | Small protein |
| 1.1.1.1 | | | |
| 1.2.1.10/ | P0A9Q7 | E. coli (Ec) | Bi-functional |
| 1.1.1.1 | | | protein |
| 1.2.1.10/ | — | T. thermosaccharolyticum | Assumed bi- |
| 1.1.1.1 | | (Tats) | functional (no |
| | | | sequence |
| | | | available) |
| 1.3.99.2/FeS | Q3EZ11 | B. thuringiensis (Bt) | 2 copies of |
| | | | 1.3.99.2, No |
| | | | annotated EtfAB |
| | | | cofactors but |
| | | | large FeS protein |
| 1.3.99.2/ | Z92974 | T. thermosaccharolyticum | 1.3.99.2 and |
| EtfAB | | (Tats) | associated |
| | | | cofactors EtfA |
| | | | and EtfB |
| BuOH | Z92974 | T. thermosaccharolyticum | Homologues of |
| operon | | (Tats) | 4.2.1.17, |
| | | | 1.3.99.2/EtfAB, |
| | | | 1.1.1.35 and |
| | | | 2.3.1.9 |

### Plasmid Construction:

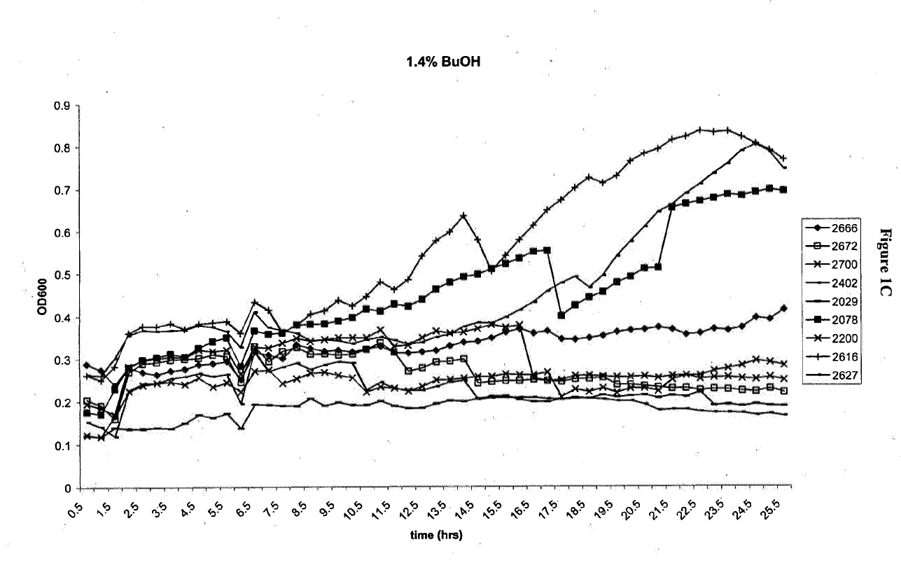

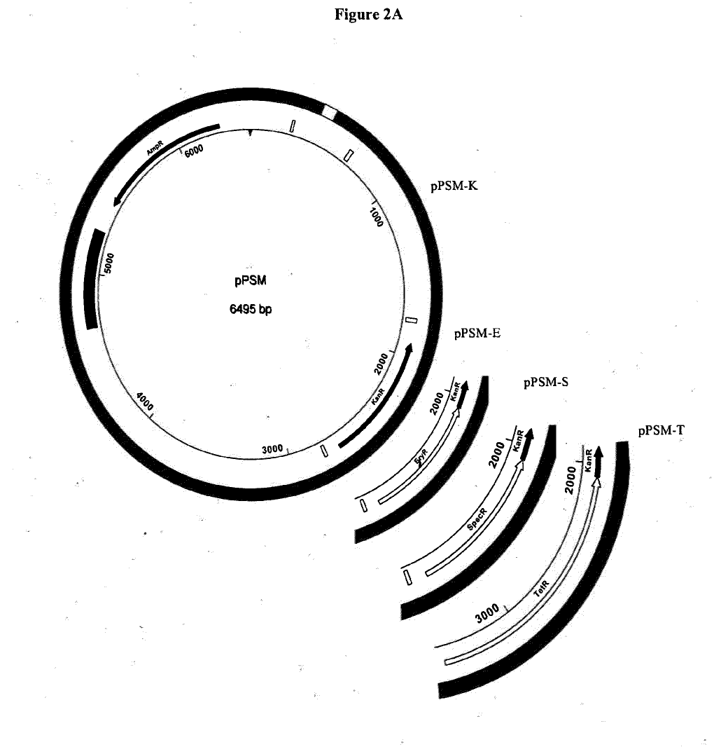

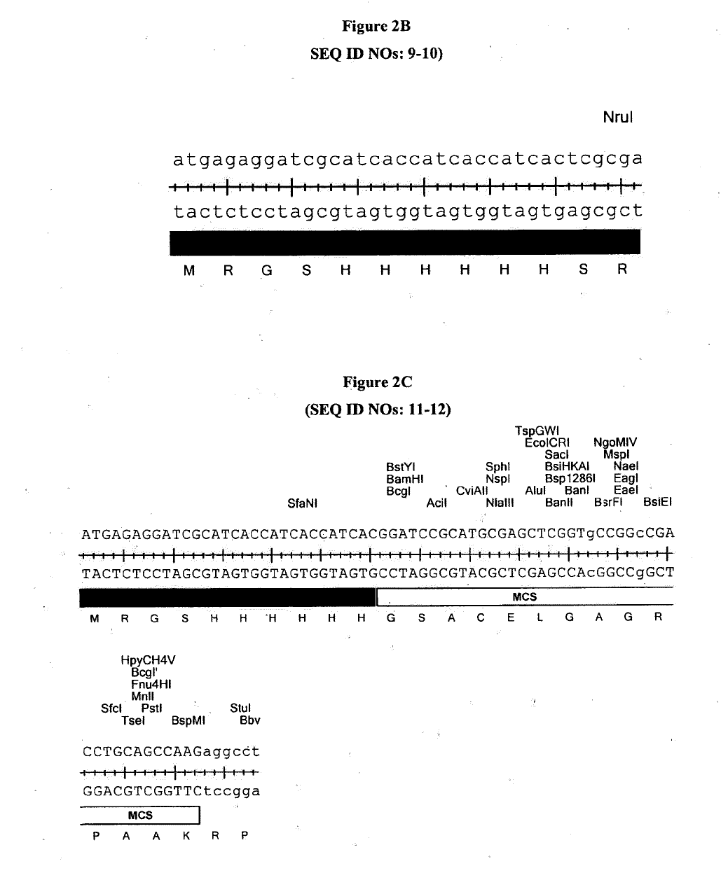

pPSM-K: Based on pUBUC, this plasmid enables the kanamycin resistance cassette to be removed and replaced by other selectable markers. First, a limited multiple cloning site from pSL1180 (GE Healthcare) was cloned in between the PstI and XbaI sites of pUBUC. Then site directed mutagenesis (Stratagene) was used to introduce a SpeI site into the start of the Kan gene using primers LJ086 and LJ087 (Table 3). The incorporation of the restriction site was confirmed by sequencing. This plasmid is designated pPSM-K. In order to create a series of vectors with a range of selectable markers, a SpeI/BglII double digest was used to remove most of the kanamycin gene. The resistance genes for erythromycin, spectinomycin and tetracycline were then amplified from Bacillus plasmids pDG641, pDG1726 and pDG1515 respectively (Bacillus stock centre), incorporating 5′ SpeI and 3′ BglII restriction sites (primers LJ094-LJ099). These were cloned into pGemT following manufacturer's guidelines and then subcloned into pPSM using the SpeI and BglII sites to create plasmids pPSM-E (erythromycin), pPSM-T (tetracycline) and pPSM-S (spectinomycin) (FIG. 2A).

pPSM-pLdhHis and pPSM-pLdhLHis: These expression vectors were created in order to N-terminally his-tag the targeted genes enabling us to determine whether the proteins can be expressed in the thermophilic strains. Plasmid pPSM-K was used as the basis for these constructs. The pLdh promoter (as described below) was amplified using primers LJ080 and LJ116, incorporating a SmaI site after the ribosome binding site to allow for gene cloning at the optimal spacing for efficient transcription. This was blunt cloned into pPSM-K. The his tag was made by annealing together oligos (LJ142 and LJ143). 100 pmol each oligo were mixed in a final volume of 20 μl in H₂O. This was incubated at 95° C. for 5 mins and then gradually cooled 70° C. to 30° C. at 1° C./min. A final cooling step of 30° C. to 4° C. over 5 mins was followed by a 4° C. hold. In order to make pPSM-pLdhHis, this annealed DNA fragment was blunt cloned into the SmaI site downstream from the pLdh promoter. This ‘short’ his tag construct has a single in-frame NruI cloning site for inserting genes (FIG. 2B). In order to increase the number of cloning sites, a ‘long’ his tag construct was made by annealing a further two oligos together (LJ152 and LJ153) which consist of a sequence of in-frame restriction sites. This fragment was then blunt cloned into the pPSM-pLdhHis NruI site to create pPSM-pLdhLHis (FIG. 2C).

### Promoters

The SecDF promoter (pSec) and Ldh promoter (pLdh) from Geobacillus stearothermophilus, and the beta-galactosidase promoter (pGal) from Geobacillus kaustophilus were chosen as promoters as they should be constitutively expressed. pLdh and pSec promoters were PCR amplified from gDNA, A-tailed and ligated into pGem-T (Promega). A NdeI restriction enzyme site was engineered into the 3′-ends of both pSec and pLdh therefore allowing the start codon of cloned genes to lie at optimal spacing from the ribosome binding site.

pGal was cloned directly into plasmid pPSM-K. PCR was carried out using PNK phosphorylated primers (protocol outlined by NEB). Labelled primers were then used in a standard PCR reaction. Phosphorylated pGal was cloned into pPSM-K that had been digested with StuI and then dephosphorylated using Antarctic Phosphatase (protocol outlined by NEB). The pGal promoter incorporates a SmaI site downstream from the ribosome binding site to allow the gene targets to be cloned directly in with the optimal spacing.

All promoters were tested for activity by cloning them upstream of a thermostable β-galactosidase gene. The β-galactosidase gene was PCR amplified from G. kaustophilus DSM7263 gDNA and cloned into pPSM-K. Reporter plasmids were transformed into G. thermoglucosidasius and colonies grown in media containing x-gal. The presence of a blue colouring indicated the promoter was active.

### Subcloning

Blunt cloning protocol:

Unless otherwise described, all blunt cloning was carried out using the following protocol. 50 ng of plasmid DNA was incubated with varying molar ratios of insert DNA in quick ligation buffer (Promega) with 0.5 μl blunt cutting restriction enzyme (e.g. StuI or SmaI) at the optimal temperature for the enzyme for 30 mins. 1 μl of T4 DNA ligase was then added and the ligation mix incubated at 22° C. for 3 hours. The volume was made up to 20 μl in 1× restriction enzyme buffer and a further 0.5 μl of the blunt cutting enzyme added. Samples were incubated for 1 hour at the optimal restriction enzyme temperature. 5 μl was used directly to transform 50 μl E. coli competent cells.

In all cases, after overnight incubations at 37° C., transformed E. coli colonies were checked by colony PCR using either insert specific primers or primer pairs that allow for insert direction determination. Standard PCR reactions were carried out using 10 μl of a boiled colony solution (a single colony resuspended in 500 μl H₂O and boiled for 10 mins at 95° C.) as the template. Samples were run on TAE agarose gels and positive clones identified by the presence of PCR products of the expected size. Positive clones were grown in overnight cultures and the plasmids extracted using a miniprep kit (Promega). All clones were confirmed by sequencing (GATC-Biotech).

### Constructs Made:

Bt1.2.1.10: This gene was amplified and cloned into pGemT. The insert was digested out using NdeI/SacfI and then ligated into pGemT carrying the pSec promoter. The sequence of the pSec-Bt12110 cassette was confirmed by sequencing. This cassette was then PCR amplified out of pGemT and blunt cloned into pPSM-pLdhLHis StuI site.

Ec 1.2.1.10: The pSec-Ec 12110 cassette was made as described for Bt 12110 above. This was also blunt cloned into pPSM-pLdhLHis StuI site.

Bt1.3.99.2/FeS: Both 1.3.99.2 homologues occur in an operon with the FeS gene which is predicted to fulfil a function similar to the EtfAB proteins in Tats. A PCR fragment containing the FeS gene is blunt cloned directly into the StuI site of pPSM-pLdhLHis. The PCR fragment incorporates a 3′ extension containing a ribosome binding site and a SmaI blunt cloning site for the subsequent cloning of the two 1.3.99.2 genes. This creates an artificial Bt1.3.99.2/FeS operon.

Tats 1.3.99.2/EtfAB: This fragment of the BCS operon was PCR amplified and blunt cloned directly into pPSM-pLdhHis NruI site. Inserts in the correct orientation were identified by directional colony PCR.

Tats Butanol operon: The first 6 genes of this operon were PCR amplified, incorporating a 5′ XhoI site. The PCR fragment was blunt cloned into the pPSM-K PmlI site to create plasmid pPSM-BCS. The pLdh promoter and his tag were added by digesting pLdhLHis out of pPSM-pLdhLHis using XhoI and NotI. This fragment was then ligated upstream of the BCS operon to create pPSM-pLdhLHis+BCS.

Tats 1.2.1.10: There is no sequence available for this gene. Degenerate primers were designed based on a number of 1.2.1.10 homologues and used to amplify a short 3′ region. Based on the sequence of this fragment, the gene from a few strains with high identity were aligned and used to identify closely homologous regions in the 5′ of the gene. A second set of degenerate primers were then designed to this region and used to amplify up the majority of the Tats 1.2.1.10 sequence. The extreme 3′ and 5′ ends, which do not show close homology, were identified by inverse PCR. After the entire coding sequence is identified, PCR primers are designed in order to blunt clone the gene into an expression vector either with the rest of the Tats BuOH operon or into a second plasmid that is compatible for co-transformations (e.g. pNW33N)

Tats1.3.99.2/EtfAB and Bt1.2.1.10: Both genes were cloned into a single plasmid. The plasmid containing the Tats 1.3.99.2/EtfAB genes was digested with PmlI. The pSec-Bt12110 cassette was PCR amplified and blunt cloned into the PmlI site. The orientation of this insert is reverse with respect to the Tats13992/EtfAB cassette.

Artificial operons: Operons consisting of both 1.3.99.2/EtfAB and 1.2.1.10 homologues are created under the control of a single promoter (pLdh). The Tats1.3.99.2/EtfAB sequence cloned into pPSM-pLdhHis contains a 3′ extension which has a ribosome binding site and a SmaI blunt cloning site for the addition of further genes, e.g. 1.2.1.10 homologues.

TABLE 3
Sequences of oligos used for PCR amplifications

| | SEQ | | | |
|---|---|---|---|---|
| | ID | Primer | | RE |
| Target | NO | No. | Sequence 5′- 3′ | sites |
| Plasmid | | | | |
| Modifications | | | | |
| SDM oligo pair | 1 | LJ086 | GGTCCATTCACTAGTCTCATTCCCTTTTCAG | Spel |
| | 2 | LJ087 | CTGAAAAGGGAATGAGACTAGTGAATGGAC | Spel |
| | | | C | |
| Spectinomycin | 3 | LJ094 | ACTAGTgaggaggatatatttgaatac | Spel |
| cassette | 4 | LJ095 | AGATCTttataatttttttaatctgttatttaaatag | Bglll |
| Tetracycline | 5 | LJ096 | ACTAGTgaatacatcctattcacaatcg | Spel |
| cassette | 6 | LJ097 | AGATCTttagaaatccctttgagaatg | Bglll |
| Erythromycin | 7 | LJ098 | ACTAGTgaacgagaaaaatataaaacacag | Spel |
| cassette | 8 | LJ099 | AGATCTttacttattaaataatttatagctattg | Bglll |
| His tags | | | | |
| “Short” tag | 9 | LJ142 | ATGAGAGGATCGCATCACCATCACCATCACt | Nrul |
| | | | cgcga | |
| | 10 | LJ143 | tcgcgaGTGATGGTGATGGTGATGCGATCCTC | Nrul |
| | | | TCAT | |
| “Long” tag | 11 | LJ152 | GGATCCGCATGCGAGCTCGGTgCCGGcCGA | Multiple |
| | | | CCTGCAGCCAAGaggcct | |
| | 12 | LJ153 | aggcctCTTGGCTGCAGGTCGgCCGGcACCGA | Multiple |
| | | | GCTCGCATGCGGATCC | |
| Promoters | | | | |
| pLdh | 13 | LJ080 | CATCCGCTATATATTAACGTGGGTGC | None |
| | 14 | LJ081 | CCACCGTTGTTTTTcatatgATTCATCCTCCC | Ndel |
| | 15 | LJ116 | CCCGGGTTCATCCTCCCTCAATATAATGC | Smal |
| pSec | 16 | LJ027 | cctaggGTGCGATGAGTTTCGTCTTC | Avrll |
| | 17 | LJ028 | catatgCCTTAAGATTCCTCCTTCAAC | Ndel |
| pGal | 18 | LJ100 | GTGATTTTACTGTATCCTTC | None |
| | 19 | LJ101 | CCCGGGTATTCCCCCTAGCTAATTTTCG | Smal |
| Genes | | | | |
| B-Galactosidase | 20 | LJ108 | ATGAATGTGTTATCCTCAATTTGTTACG | None |
| | 21 | LJ109 | CTAAACCTTCCCGGCTTCATCATGC | None |
| Bt 1.3.99.2 | 22 | LJ025 | catATGGAGGCGGAGTATATGAAC | Ndel |
| | 23 | LJ026 | ctcgagTTAggtaccTAAAGCACGGCTAATAACA | Xhol/ |
| | | | AG | Kpnl |
| Bt 1.3.99.2 | 24 | LJ165 | ATGGAGGCGGAGTATATGAACTTTCG | None |
| both genes | 25 | LJ166 | CTACTTCGTTAACATACGAGAAATTAC | None |
| Bt FeS | 26 | LJ163 | ATGAATAGCTTACTGATCATTAATTGGC | None |
| | 27 | LJ164 | cccgggTGCAGATTACATCGCTTCTTTC | Smal |
| Tats 1.3.99.2 | 28 | LJ161 | ATGGACTTTTCATTAACAAAGGAGC | None |
| +EtfAB | 29 | LJ162 | cccgggACACTCCTTTTTATTTTTATTAAGGTC | Smal |
| Ec 1.2.1.10 | 30 | LJ033 | catATGGCTGTTACTAATGTCGC | Ndel |
| | 31 | LJ034 | gagctcggatccTTAcccgggAGCGGATTTTTTCGC | Sacl/Ba |
| | | | TTTTTTCTC | mHl/Xma |
| | | | | l |
| Bt 1.2.1.10 | 32 | LJ031 | catATGATGAGAAGCTTCAAACCTGATG | Ndel |
| | 33 | LJ032 | gagctcggatccTTAcccgggTTCACCTTTATATGC | Sacl/Ba |
| | | | TTCCATATAAATTTC | mHl/Xma |
| | | | | l |
| Operons | | | | |
| Tats BuOH | 34 | LJ167 | ctcgagggATGGATTTTAATAATGTTTTATTAAA | Xhol |
| | | | TAAGGATG | |
| operon | 35 | LJ168 | gttaacCTATCTTTCGACAACCATTGCTATTCC | Hpal |

RE sites = restriction enzyme sites. In most cases these are 3′ or 5′ additions. In some cases they engineer sites into existing sequences.

### Example 3

### Generation of New Strains

### Testing for Restriction-Modification System

One of the main barriers to successful transformation of host bacterial strains is the presence of restriction-modification systems. Prior to transformation attempts, strains were tested for restriction activity against both the plasmids being used in this work and (as a control), unmethylated λ gDNA (NEB). Protoplasts were prepared from overnight cultures. Cell pellets were resuspended in protoplasting medium (LB containing 0.59 mM MgSO₄, 0.91 mM CaCl₂, 0.04 mM FeSO₄, 10% lactose and 10 mM MgCl₂) and lysozyme to 10 μg/ml and were incubated at 55° C. with slow shaking (130 rpm) for 30 mins. Protoplasts were diluted 1:1 with more protoplasting medium and then lysed using 2 ml TEMK per gram of cell pellet (4 mM Tris pH8.0, 10 mM EDTA, 23 μl β-mercaptoethanol and 1.25 mM KCl). Cell debris was removed by centrifugation. Genomic DNA and other nucleic acids were removed using streptomycin sulphate precipitation. Streptomycin (Sigma) was added to 1% and the lysed protoplasts were incubated on ice for 30 mins. Centrifugation pelleted the DNA and the cleared supernatant was used in restriction-modification assays.

### Restriction-Modification Assay

A reaction mix containing 1× restriction enzyme buffer 3 (NEB), 1×BSA (NEB), 5 μl lysed protoplast sample and test DNA (1 μg) in a final volume of 30 μl was incubated at 52° C. for 90 mins before being stopped by the addition of 1×DNA loading dye (Promega) and samples analysed on 1×TAE agarose gels. Controls of untreated DNA were run alongside the experimental samples. The presence of multiple bands in the treated samples was used as evidence of a restriction system active against that particular plasmid.

For G. thermoglucosidasius, if the plasmids were first pre-treated with M.HaeIII (NEB) following manufacturers' protocols; the lysed protoplasts no longer digested the substrates. Therefore prior to all transformation attempts, the plasmids described in Example 2 were methylated in vivo by transformation into an E. coli strain harbouring the m.HaeIII gene cloned into a low copy number plasmid, pCL1920 (according to protocols, Elsworth WO 01/85966).

### Transformation of Geobacillus

G. thermoglucosidasius was grown on TSB plates overnight at 60° C. Several colonies were then used to inoculate 50 ml TSB. The cells were grown until they reached an OD of 0.6-0.8.

The cells were prepared for electroporation at room temperature. They were spun down at 6000 g for 10 mins at room temperature and then washed once in 10 ml THC buffer (272 mM trehalose, 8 mM Hepes, 50 mM CaCl₂ pH7.5 with KOH) once in 5 ml THC, and once in 5 m1 TH buffer (as THC without the CaCl₂) before finally resuspending in 400 μl TH buffer. Cells were either snap frozen and stored at −80° C. or used straight away.

Methylated DNA (500 ng) was added directly to the cells and incubated at room temperature for 5 mins before electroporating in 4 mm cuvettes. Parameters for electroporation were 3000V, 200Ω and 250 μF. Immediately after electroporation, 800 μl pre-warmed TSB containing 2.5% glucose was added and the cells were incubated at 52° C. for 1 hour before 250 μl was plated on TSB+12 ng/μl kan, and incubated for 24-48 hours at 52° C.

The plasmids described in Example 2 have been transformed into Geobacillus thermoglucosidasius (Table 4).

TABLE 4
New Geobacillus strains generated

| Genes added | Strain number | Notes |
|---|---|---|
| Bt12110 | GBL4136 and 4137 | His tagged |
| Ec12110 | GBL4138 | His tagged |
| Tats13992/EtfAB | GBL4131 | His tagged 1.3.99.2 |
| Tats13992/EtfAB + Bt12110 | GBL4139 and 4140 | His tagged 1.3.99.2 |

In some cases it is desirable to transform multiple plasmids into a single strain. These plasmids may be carrying different sets of genes, which when transformed into a single cell can be used to complete the butanol biosynthetic pathway. Plasmid pNW33N is compatible with pUBUC based plasmids, including pPSM-K and pUB-MCS. pNW33N has been modified to incorporate a more extensive multiple cloning site (plasmid pNW-MCS). The EcoRI-HindIII fragment of the pSL1180 multiple cloning site was digested out and ligated into pNW33N. This can be used for cloning of the genes mentioned in the previous examples or other genes that are predicted to play a role in butanol production. pUBUC was also modified to incorporate a more extensive multiple cloning site (pUB-MCS). In this case, the HindIII-BamHI fragment from pSL1180 was ligated into pUBUC.

In order to make double transformants, Geobacillus was transformed with one of the plasmids e.g. pUB-MCS, selected for using kanamycin. These cells were then grown in TSB containing 12 μg/ml kanamycin to an OD of 0.6-0.8. The cells were prepared in THC and TH buffers as described previously. The second plasmid e.g. pNW-MCS was then electroporated into the strain using the same parameters as used previously. The presence of this second plasmid was selected for using 15 μg/ml chloramphenicol. Double transformants were able to grow on media containing both kanamycin and chloramphenicol. Strain L04 contains both empty vectors pUB-MCS and pNW-MCS.

### Example 4

### Characterisation of New Strains

### BuOH Production

To test for the production of butanol, transformed cells were plated onto TSB and grown overnight at 52° C. From these plates, colonies were inoculated into 5 ml TSB and the cultures grown until turbid. A 5% inoculum was then either used in minimal media containing 1% glucose as the carbon source or in TSB containing 1% glucose. Cultures were either left shaking aerobically at 52° C., in a static aerobic incubator or in a static anaerobic incubator until growth was observed. 1 ml samples were taken and centrifuged for GC analysis.

### Protein Expression

Of the genes inserted into the expression plasmids, most are from mesophilic organisms. The only thermophilic proteins come from Tats. This may affect enzyme activity when grown at 52° C. Additionally the codon usage varies between organisms. For these reasons, the his-tag constructs were developed to enable us to check for protein expression.

Overnight cultures were grown at 52° C. either aerobically with shaking or anaerobically without. Pellets were resuspended in resuspension buffer containing 6M urea (20 mM Tris pH8.0, 100 mM NaCl, 5% glycerol, 1 μM Pepstatin A, 6M Urea) and lysozyme was added to a final concentration of 0.75 mg/ml. Samples were incubated at room temperature for 30 mins before the addition of SDS loading buffer (6× stock is 30% β-mercaptoethanol, 12% SDS, 10% glycerol, 0.1% bromophenol blue and 440 mM Tris pH6.8). Prior to loading onto 12.5% SDS-PAGE gels, the samples were boiled for 10 mins at 99° C. His-tagged proteins were visualised following Western blotting onto PVDF membrane by incubation with an anti-RGS his tag primary antibody (Qiagen) and colorimetric detection using an anti-mouse secondary antibody and BCIP/NBT substrate (Sigma).

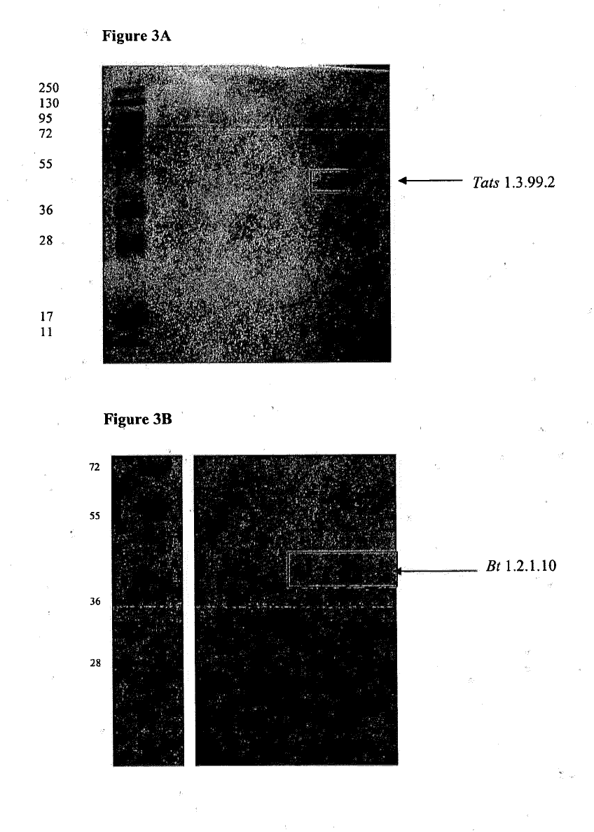

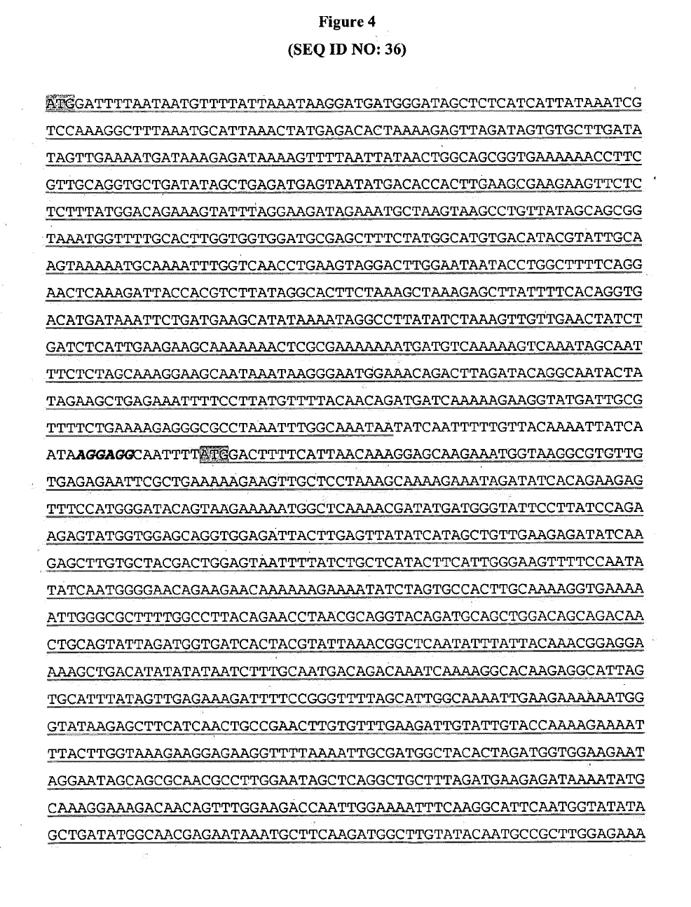

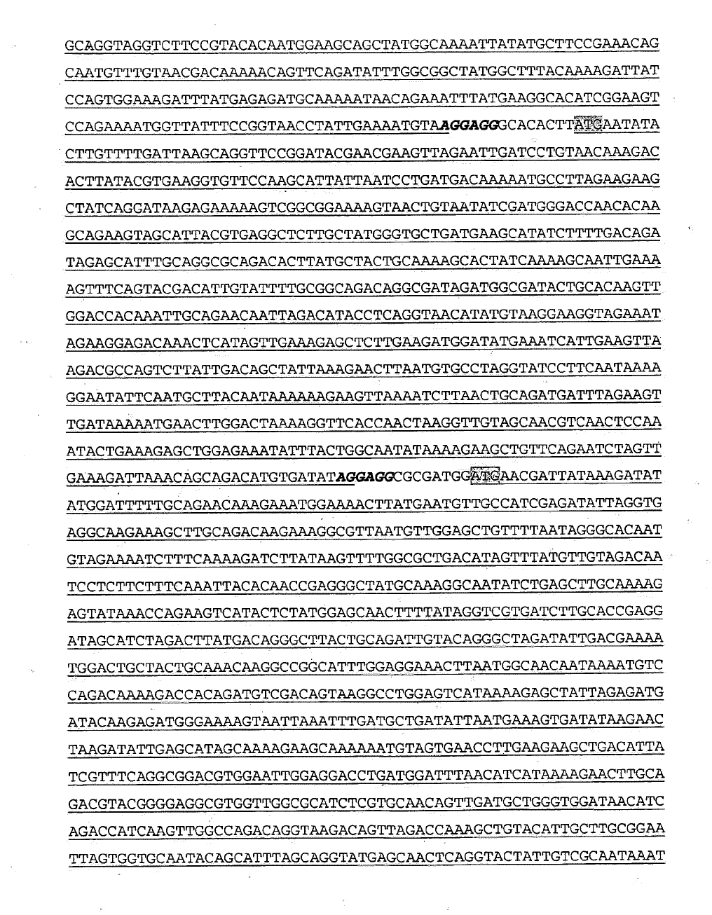

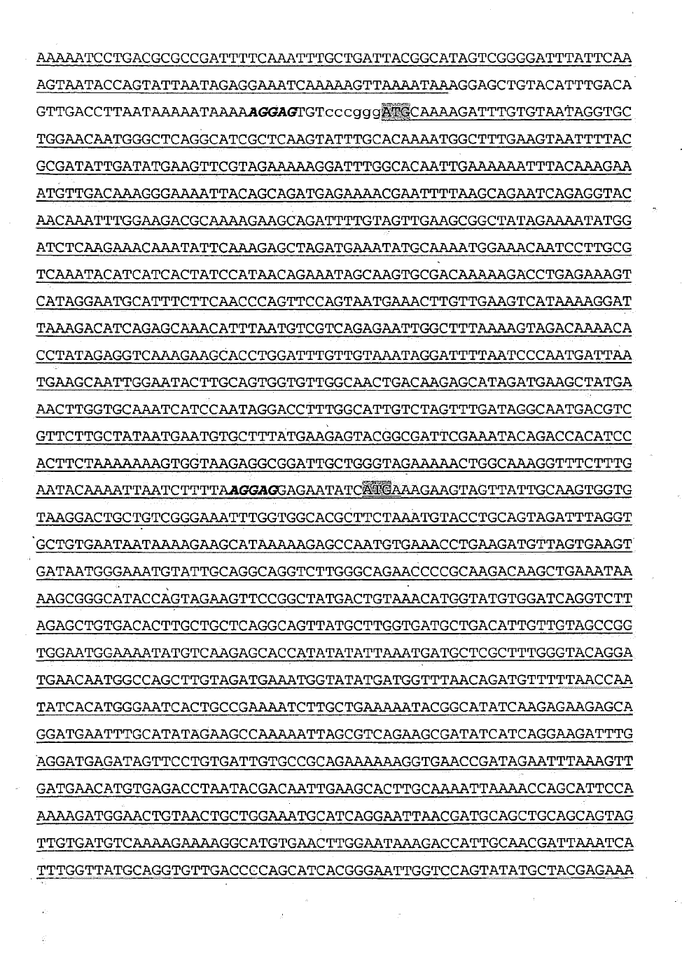

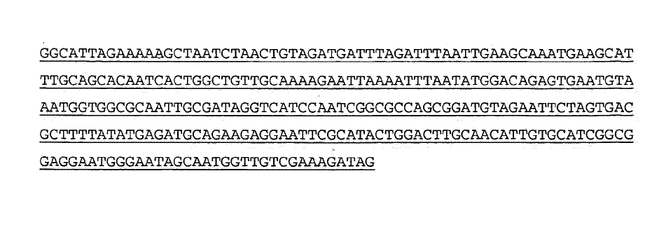

Expression of Bt1.2.1.10, Ec1.2.1.10 and Tats1.3.99.2 proteins have all been confirmed by Western blotting (FIGS. 3A and B). Codon usage could be optimised in order to increase expression levels and stability of the proteins.

For the entire Tats butanol operon to be functional, expression of these genes may need to be induced anaerobically.

### Codon Optimisation

Codon usage tables are available for sequenced strains. Strains which are not completely sequenced but which have some nucleotide sequences in the database also have limited codon tables. These can be used to re-code proteins of interest.

### SEQUENCE LISTING FREE TEXT

<210> 1 <223> PCR primer for SDM
<210> 2 <223> PCR primer for SDM
<210> 3 <223> PCR primer for spectinomycin cassette
<210> 4 <223> PCR primer for spectinomycin cassette
<210> 5 <223> PCR primer for tetracycline cassette
<210> 6 <223> PCR primer for tetracycline cassette
<210> 7 <223> PCR primer for erythromycin cassette
<210> 8 <223> PCR primer for Erythromycin cassette

### <210> 9 <223> Short HIS tag

### <210> 10 <223> Short HIS tag

### <210> 11 <223> Long HIS tag

### <210> 12 <223> Long HIS tag

<210> 13 <223> PCR primer for pLdh promoter
<210> 14 <223> PCR primer for pLdh promoter
<210> 15 <223> PCR primer for pLdh promoter
<210> 16 <223> PCR primer for pSec promoter
<210> 17 <223> PCR primer for pSec promoter
<210> 18 <223> PCR primer for pGal promoter
<210> 19 <223> PCR primer for pGal promoter
<210> 20 <223> PCR primer for B-galactosidase gene
<210> 21 <223> PCR primer for B-galactosidase gene
<210> 22 <223> PCR primer for Bt 1.3.99.2 gene
<210> 23 <223> PCR primer for Bt 1.3.99.2 gene
<210> 24 <223> PCR primer for Bt 1.3.99.2 (both) genes
<210> 25 <223> PCR primer for Bt 1.3.99.2 (both) genes
<210> 26 <223> PCR primer for Bt FeS gene
<210> 27 <223> PCR primer for Bt FeS gene
<210> 28 <223> PCR primer for Tats 1.3.99.2+EtfAB genes
<210> 29 <223> PCR primer for Tats 1.3.99.2+EtfAB genes
<210> 30 <223> PCR primer for Ec 1.2.1.10 gene
<210> 31 <223> PCR primer for Ec 1.2.1.10 gene
<210> 32 <223> PCR primer for Bt 1.2.1.10 gene
<210> 33 <223> PCR primer for Bt 1.2.1.10 gene
<210> 34 <223> PCR primer for Tats butanol operon
<210> 35 <223> PCR primer for Tats butanol operon

1. A process of producing butanol comprising:
culturing a recombinant butanol-tolerant thermophilic Bacillaceae which comprises one or more heterologous nucleic acid molecules which encode one or more butanol biosynthetic pathway enzymes selected from the group consisting of enzymes which catalyse one or more of the following reactions:
V. butyryl-CoA to butyraldehyde
VI. butyraldehyde to 1-butanol
IV. crotonyl-CoA to butyryl-CoA
III. 3-hydroxybutyryl-CoA to crotonyl-CoA
II. acetoacetyl-CoA to 3-hydroxybutyryl-CoA
I. acetyl-CoA to acetoacetyl-CoA
together with an appropriate substrate; and
harvesting the butanol from the culture medium.

2. A process as claimed in claim 1, wherein the thermophilic Bacillaceae is a Geobacillus or a Ureibacillus.

3. A method for producing a recombinant Bacillaceae comprising introducing one or more nucleic acid molecules which encode one or more butanol biosynthetic pathway polypeptides into a thermophilic butanol-tolerant Bacillaceae.

4. A method as claimed in claim 3, wherein the one or more butanol biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol.

5. A method as claimed in claim 3, wherein the introduction of the one or more nucleic acid molecules which encode one or more butanol biosynthetic pathway polypeptides results in a recombinant Bacillaceae which is capable of producing butanol.

6. A method as claimed claim 3, wherein the thermophilic Bacillaceae into which the nucleic acid molecule(s) are introduced is a non-butanol producing Bacillaceae.

7. A method for producing a recombinant Bacillaceae comprising introducing one or more nucleic acid molecules which encode one or more butyrate biosynthetic pathway polypeptides into a thermophilic butanol-tolerant Bacillaceae.

8. A method as claimed in claim 7, wherein the one or more butyrate biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyryl phosphate
VI. butyryl phosphate to butyrate.

9. A method as claimed in claim 7, wherein the introduction of the one or more nucleic acid molecules which encode one or more butyrate biosynthetic pathway polypeptides results in a recombinant Bacillaceae which is capable of producing butyrate.

10. A method as claimed in claim 7, wherein the thermophilic Bacillaceae into which the nucleic acid molecule(s) are introduced is a non-butyrate producing Bacillaceae.

11. A method for producing a recombinant thermophilic Bacillaceae which is capable of producing butanol, comprising:
(a) selecting a population of thermophilic Bacillaceae
(i) which do not produce butanol,
(ii) which are butanol-tolerant, and
(iii) which have one or more genes encoding butanol biosynthetic pathway polypeptides endogenously present in their genome,
wherein the butanol biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol
and
(b) introducing into a thermophilic Bacillaceae from the selected population nucleic acid molecules coding for one or more of those enzymes I-VI which were not endogenously present in the genome of the thermophilic Bacillaceae, in order to produce a recombinant thermophilic Bacillaceae which is capable of producing butanol.

12. A method for producing a recombinant thermophilic Bacillaceae which is capable of producing butyrate, comprising:
(a) selecting a population of thermophilic Bacillaceae
(i) which do not produce butyrate,
(ii) which are butyric acid tolerant, and
(iii) which have one or more genes encoding butyrate biosynthetic pathway polypeptides endogenously present in their genome,
wherein the butyrate biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyryl phosphate
VI. butyryl phosphate to butyrate
and
(b) introducing into a thermophilic Bacillaceae from the selected population nucleic acid molecules coding for one or more, of those enzymes I-VI which were not endogenously present in the genome of the thermophilic Bacillaceae, in order to produce a recombinant thermophilic Bacillaceae which is capable of producing butyrate.

13. A method as claimed in claim 3, wherein:
(A) one or more nucleic acid molecules which encode 1, 2, 3, 4, 5 or 6 of enzymes I-VI are introduced into the thermophilic Bacillaceae,
(B) one or more nucleic acid molecules which encode enzymes I-II, I-III, I-IV, I-V or I-VI are introduced into the thermophilic Bacillaceae,
(C) one or more nucleic acid molecules which encode enzymes II-III, II-IV, II-V or II-VI are introduced into the thermophilic Bacillaceae,
(D) one or more nucleic acid molecules which encode enzymes III-IV, III-V or III-VI are introduced into the thermophilic Bacillaceae,
(E) one or more nucleic acid molecules which encode enzymes IV-V or IV-VI are introduced into the thermophilic Bacillaceae,
(F) one or more nucleic acid molecules which encode enzymes V-VI are introduced into the thermophilic Bacillaceae, or
(G) wherein one or more nucleic acid molecules which encode enzyme VI are introduced into the thermophilic Bacillaceae.

14. A method as claimed in claim 3, wherein the thermophilic Bacillaceae is one which has endogenous genes coding for 1, 2, 3, 4, 5 or 6 of enzymes I-VI.

15. A method as claimed in claim 3, wherein the Bacillaceae have:
(A) endogenous genes that encode enzymes I-II, I-III, I-IV, I-V or I-VI,
(B) endogenous genes that encode enzymes II-III, II-IV, II-V or II-VI,
(C) endogenous genes that encode enzymes III-IV, III-V or III-VI,
(D) endogenous genes that encode enzymes IV-V or IV-VI,
(E) endogenous genes that encode enzymes V-VI,
(F) an endogenous gene that encodes enzyme VI, or
(G) wherein the thermophilic Bacillaceae is one which has endogenous genes coding for 1, 2, 3, 4, or 5 of enzymes I-VI and the one or more nucleic acid molecules which are introduced into the thermophilic Bacillaceae encode one or more of those enzymes I-VI which are not endogenously present in the thermophilic Bacillaceae.

16. A method as claimed in claim 3, wherein a single enzyme is used to catalyse two of reactions I-VI, in place of two separate enzymes.

17. A method as claimed in claim 16, wherein a single enzyme is used to catalyse reactions V+VI.

18. A method as claimed in claim 3, wherein the Bacillaceae have endogenous genes which encode enzymes I, II and III and nucleic acid molecules coding for enzymes which catalyse reactions IV, V and VI are introduced into the thermophilic Bacillaceae.

19. A method as claimed in claim 18, wherein one enzyme is used to catalyse reactions V and VI.

20. A method as claimed in claim 3, wherein nucleic acid molecules coding for enzymes EC 1.2.1.10 and EC 1.3.99.2, and optionally coding for cofactors EtfA and EtfB, or equivalents are introduced into a thermophilic Bacillaceae which has endogenous genes coding for enzymes which are capable of catalysing reactions I-III.

21. A method for producing a recombinant Bacillaceae comprising introducing one or more nucleic acid molecules which encode one or more butanol and/or butyrate biosynthetic pathway polypeptides into a thermophilic butanol-tolerant Bacillaceae.

22. A method as claimed in claim 21, wherein the one or more butanol and/or butyrate biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol
VII. butyryl-CoA to butyryl phosphate
VIII. butyryl phosphate to butyrate.

23. A method as claimed in claim 21, wherein the introduction of the one or more nucleic acid molecules which encode one or more butanol and/or butyrate biosynthetic pathway polypeptides results in a recombinant Bacillaceae which is capable of producing butanol and butyrate.

24. A method as claimed in claim 21, wherein the thermophilic Bacillaceae is a non-butanol producing Bacillaceae.

25. A method as claimed in claim 21, wherein the thermophilic Bacillaceae is a non-butyrate producing Bacillaceae.

26. A method for producing a recombinant thermophilic Bacillaceae which is capable of producing butanol and butyrate, comprising:
(a) selecting a population of thermophilic Bacillaceae
(i) which do not produce butanol or butyrate,
(ii) which are butanol-tolerant and butyric acid tolerant, and
(iii) which have one or more genes encoding butanol and/or butyrate biosynthetic pathway polypeptides endogenously present in their genome,
wherein the butanol and/or butyrate biosynthetic pathway polypeptides are selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol
VII. butyryl-CoA to butyryl phosphate
VIII. butyryl phosphate to butyrate
and
(b) introducing into a thermophilic Bacillaceae from the selected population nucleic acid molecules coding for one or more of those enzymes I-VIII which were not endogenously present in the genome of the thermophilic Bacillaceae, in order to produce a recombinant thermophilic Bacillaceae which is capable of producing butanol and butyrate.

27. A method as claimed in claim 21, wherein:
(A) one or more nucleic acid molecules which encode 1, 2, 3, 4, 5, 6, 7 or 8 of enzymes I-VIII are introduced into the thermophilic Bacillaceae,
(B) one or more nucleic acid molecules which encode enzymes I-II, I-III, I-IV, I-V, I-VI, I-VII or I-VIII are introduced into the thermophilic Bacillaceae,
(C) one or more nucleic acid molecules which encode enzymes II-III, II-IV, II-V, II-VI, II-VII or II-VIII are introduced into the thermophilic Bacillaceae,
(D) one or more nucleic acid molecules which encode enzymes III-IV, III-V, III-VI, III-VII or III-VIII are introduced into the thermophilic Bacillaceae,
(E) one or more nucleic acid molecules which encode enzymes IV-V, IV-VI, IV-VII or IV-VIII are introduced into the thermophilic Bacillaceae,
(F) one or more nucleic acid molecules which encode enzymes V-VI, V-VII or V-VIII are introduced into the thermophilic Bacillaceae,
(G) one or more nucleic acid molecules which encode enzymes VI-VII or VI-VIII are introduced into the thermophilic Bacillaceae,
(H) one or more nucleic acid molecules which encode enzymes VII-VIII are introduced into the thermophilic Bacillaceae, or
(I) one or more nucleic acid molecules which encode enzyme VIII are introduced into the thermophilic Bacillaceae.

28. A method as claimed in claim 21, wherein:
(A) the thermophilic Bacillaceae is one which has endogenous genes coding for 1, 2, 3, 4, 5, 6, 7 or 8 of enzymes I-VIII,
(B) the Bacillaceae have endogenous genes that encode enzymes I-II, I-III, I-IV, I-V, I-VI, I-VII or I-VIII,
(C) the Bacillaceae have endogenous genes that encode enzymes II-III, II-IV, II-V, II-VI, II-VII or II-VIII,
(D) the Bacillaceae have endogenous genes that encode enzymes III-IV, III-V, III-VI, III-VII or III-VIII,
(E) the Bacillaceae have endogenous genes that encode enzymes IV-V, IV-VI, IV-VII or IV-VIII,
(F) the Bacillaceae have endogenous genes that encode enzymes V-VI, V-VII or V-VIII,
(G) the Bacillaceae have an endogenous gene that encode enzymes VI-VII or VI-VIII,
(H) the Bacillaceae have an endogenous gene that encode enzymes VII-VIII,
(I) the Bacillaceae have an endogenous gene that encodes enzyme VIII, or
(J) the thermophilic Bacillaceae is one which has endogenous genes coding for 1, 2, 3, 4, 5, 6 or 7 of enzymes I-VIII and the one or more nucleic acid molecules which are introduced into the thermophilic Bacillaceae encode one or more of those enzymes I-VIII which are not endogenously present in the thermophilic Bacillaceae.

29. A method as claimed in claim 3, wherein one or more nucleic acid molecules which encode an enzyme which catalyses the following reaction IX are introduced into the thermophilic Bacillaceae in place of one or more nucleic acid molecules which encode an enzyme which catalyses reaction III:
IX (3S)-3-hydroxyacyl-CoA to trans-2(or 3)-enoyl-CoA.

30. A method as claimed in claim 3, wherein one or more nucleic acid molecules which encode an enzyme which catalyses the following reaction X are introduced into the thermophilic Bacillaceae in place of one or more nucleic acid molecules which encode an enzyme which catalyses reaction IV:
X acyl-CoA to trans-didehydroacyl-CoA.

31. A method as claimed in claim 3, which additionally comprises producing a master cell bank and/or working cell bank of the recombinant Bacillaceae, and growing a culture from the master and/or working cell banks.

32. A method as claimed in claim 3, wherein the method further comprises storing aliquots of the recombinant Bacillaceae at a temperature of 5° C. or lower; and subsequently growing the recombinant Bacillaceae on a solid media or in a liquid media.

33. A method as claimed in claim 3, wherein:
(A) the Bacillaceae is a Gram +ve Bacillaceae,
(B) the Bacillaceae is a Gram −ve Bacillaceae,
(C) the Bacillaceae is an aerobic Bacillaceae,
(D) the Bacillaceae is an anaerobic Bacillaceae or
(E) the Bacillaceae is Anoxybacillus, Bacillus, Brevibacillus, Paenibacillus, Hydrogenophilus, Geobacillus or Ureibacillus.

34. A method as claimed in claim 33, wherein the Bacillaceae is Geobacillus or Ureibacillus.

35. A method as claimed in claim 34, wherein the Bacillaceae is Geobacillus.

36. A method as claimed in claim 35, wherein the Geobacillus is Geobacillus thermoglucosidasius, Geobacillus kaustophilus, Geobacillus subterraneus, Geobacillus uzenensis, Geobacillus thermocatenulatus, Geobacillus thermodenitrificans, Geobacillus thermoleovorans, Geobacillus thermantarticus, Geobacillus pallidus, Geobacillus toebii, Geobacillus caldoxylosilyticus, Geobacillus tropicalis or Geobacillus stearothermophilus.

37. A method as claimed in claim 36, wherein the Geobacillus is Geobacillus thermoglucosidasius, Geobacillus stearothermophilus or Geobacillus thermodenitrifcans.

38. A recombinant butanol-tolerant Geobacillus or Ureibacillus which comprises one or more heterologous nucleic acid molecules which encode one or more butanol biosynthetic pathway enzymes selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol.

39. A recombinant Geobacillus or Ureibacillus as claimed in claim 38 which is capable of producing butanol.

40. A recombinant butanol-tolerant Geobacillus or Ureibacillus which comprises one or more heterologous nucleic acid molecules which encode one or more butyrate biosynthetic pathway enzymes selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyryl phosphate
VI. butyryl phosphate to butyrate.

41. A recombinant Geobacillus or Ureibacillus as claimed in claim 40 which is capable of producing butyrate.

42. A recombinant Geobacillus or Ureibacillus as claimed in claim 38, wherein:
(A) the recombinant Geobacillus or Ureibacillus comprises one or more heterologous nucleic acid molecules which encode 1, 2, 3, 4, 5 or 6 of enzymes I-VI,
(B) the recombinant Geobacillus or Ureibacillus comprises one or more heterologous nucleic acid molecules which encode enzymes I-II, I-III, I-IV, I-V or I-VI,
(C) the recombinant Geobacillus or Ureibacillus comprises one or more heterologous nucleic acid molecules which encode enzymes II-III, II-IV, II-V or II-VI,
(D) the recombinant Geobacillus or Ureibacillus comprises one or more heterologous nucleic acid molecules which encode enzymes III-IV, III-V or III-VI,
(E) the recombinant Geobacillus or Ureibacillus comprises one or more heterologous nucleic acid molecules which encode enzymes IV-V or IV-VI,
(F) the recombinant Geobacillus or Ureibacillus comprises one or more heterologous nucleic acid molecules which encode enzymes V-VI, or
(G) the recombinant Geobacillus or Ureibacillus comprises one or more heterologous nucleic acid molecules which encode enzyme VI.

43. A recombinant Geobacillus or Ureibacillus as claimed in claim 38, wherein:
(A) the Geobacillus or Ureibacillus is one which has endogenous genes coding for 1, 2, 3, 4, 5 or 6 of enzymes I-VI,
(B) the Geobacillus or Ureibacillus is one which has endogenous genes that encode enzymes I-II, I-III, I-IV, I-V or I-VI,
(C) the Geobacillus or Ureibacillus is one which has endogenous genes that encode enzymes II-III, II-IV, II-V or II-VI,
(D) the Geobacillus or Ureibacillus is one which has endogenous genes that encode enzymes III-IV or III-V or III-VI,
(E) the Geobacillus or Ureibacillus is one which has endogenous genes that encode enzymes IV-V or IV-VI,
(F) the Geobacillus or Ureibacillus is one which has endogenous genes that encode enzymes V-VI,
(G) the Geobacillus or Ureibacillus is one which has an endogenous gene that encodes enzyme VI,
(H) the Geobacillus or Ureibacillus is one which has endogenous genes coding for 1, 2, 3, 4, or 5 of enzymes I-VI and one or more heterologous nucleic acid molecules which encode one or more of those enzymes I-VI which were not endogenously present in the Geobacillus or Ureibacillus,
(I) the Geobacillus or Ureibacillus have endogenous genes which encode enzymes I, II and III and one or more heterologous nucleic acid molecules coding for enzymes which catalyse reactions IV, V and VI,
(J) one enzyme is used to catalyse reactions V and VI, or
(K) heterologous nucleic acid molecules coding for enzymes (i) EC 1.2.1.10 and (ii) EC 1.3.99.2, and optionally coding for cofactors EtfA and EtfB, or equivalents, or 1.3.1.44 are present in a Geobacillus or Ureibacillus which has endogenous genes coding for enzymes which are capable of catalysing reactions I-III.

44. A recombinant butanol-tolerant Geobacillus or Ureibacillus which comprises one or more heterologous nucleic acid molecules which encode one or more butanol and/or butyrate biosynthetic pathway enzymes selected from the group consisting of enzymes which catalyse one or more of the following reactions:
I. acetyl-CoA to acetoacetyl-CoA
II. acetoacetyl-CoA to 3-hydroxybutyryl-CoA
III. 3-hydroxybutyryl-CoA to crotonyl-CoA
IV. crotonyl-CoA to butyryl-CoA
V. butyryl-CoA to butyraldehyde
VI. butyraldehyde to 1-butanol
VII. butyryl-CoA to butyryl phosphate
VIII. butyryl phosphate to butyrate.

45. A recombinant Geobacillus or Ureibacillus as claimed in claim 44, which is capable of producing butanol and butyrate.

46. A recombinant Geobacillus or Ureibacillus as claimed in claim 44, wherein:
(A) the recombinant Geobacillus or Ureibacillus comprises one or more heterologous nucleic acid molecules which encode 1, 2, 3, 4, 5, 6, 7 or 8 of enzymes I-VIII,
(B) the recombinant Geobacillus or Ureibacillus comprises one or more heterologous nucleic acid molecules which encode enzymes I-II, I-III, I-IV, I-V, I-VI, I-VII or I-VIII,
(C) the recombinant Geobacillus or Ureibacillus comprises one or more heterologous nucleic acid molecules which encode enzymes II-III, II-IV, II-V, II-VI, II-VII or II-VIII,
(D) the recombinant Geobacillus or Ureibacillus comprises one or more heterologous nucleic acid molecules which encode enzymes III-IV, III-V, III-VI, III-VII or III-VIII.
(E) the recombinant Geobacillus or Ureibacillus comprises one or more heterologous nucleic acid molecules which encode enzymes IV-V, IV-VI, IV-VII or IV-VIII,
(F) the recombinant Geobacillus or Ureibacillus comprises one or more heterologous nucleic acid molecules which encode enzymes V-VI, V-VII or V-VIII,
(G) the recombinant Geobacillus or Ureibacillus comprises one or more heterologous nucleic acid molecules which encode enzymes VI-VII or VI-VIII,
(H) the recombinant Geobacillus or Ureibacillus comprises one or more heterologous nucleic acid molecules which encode enzymes VII-VIII, or
(I) the recombinant Geobacillus or Ureibacillus comprises one or more heterologous nucleic acid molecules which encode enzyme VIII.

47. A recombinant Geobacillus or Ureibacillus as claimed in claim 44, wherein:
(A) the Geobacillus or Ureibacillus is one which has endogenous genes coding for 1, 2, 3, 4, 5, 6, 7 or 8 of enzymes I-VIII,
(B) the Geobacillus or Ureibacillus is one which has endogenous genes that encode enzymes I-II, I-III, I-IV, I-V, I-VI, I-VII or I-VIII,
(C) the Geobacillus or Ureibacillus is one which has endogenous genes that encode enzymes II-III, II-IV, II-V, II-VI, II-VII or II-VIII,
(D) the Geobacillus or Ureibacillus is one which has endogenous genes that encode enzymes III-IV, III-V, III-VI, III-VII or III-VIII,
(E) the Geobacillus or Ureibacillus is one which has endogenous genes that encode enzymes IV-V, IV-VI, IV-VII or IV-VIII,
(F) the Geobacillus or Ureibacillus is one which has endogenous genes that encode enzymes V-VI, V-VII or V-VIII,
(G) the Geobacillus or Ureibacillus is one which has an endogenous gene that encode enzymes VI-VII or VI-VIII,
(H) the Geobacillus or Ureibacillus is one which has an endogenous gene that encode enzymes VII-VIII, or
(I) the Geobacillus or Ureibacillus is one which has an endogenous gene that encode enzyme VIII.

48. A recombinant Geobacillus or Ureibacillus as claimed in claim 44, wherein the Geobacillus or Ureibacillus is one which has endogenous genes coding for 1, 2, 3, 4, 5, 6 or 7 of enzymes I-VIII and one or more heterologous nucleic acid molecules which encode one or more, of those enzymes I-VIII which are not endogenously present in the Geobacillus or Ureibacillus.

49. A recombinant Geobacillus or Ureibacillus as claimed in any claim 44, which comprises a nucleic acid molecule which encodes a polypeptide which confers antibiotic resistance on the Geobacillus or Ureibacillus.

50. A recombinant Geobacillus or Ureibacillus as claimed in claim 44, which comprises a nucleic acid molecule which encodes a polypeptide which confers heavy metal resistance on the Geobacillus or Ureibacillus.

51. A recombinant Geobacillus or Ureibacillus as claimed in claim 44, which comprises a nucleic acid molecule which encodes a polypeptide which catalyses one or more of reactions I-VIII operably attached to an inducible promoter.

52. A recombinant Geobacillus or Ureibacillus as claimed in claim 44, which comprises a nucleic acid molecule which encodes a polypeptide which catalyses one or more of reactions I-VIII operably attached to a constitutive promoter.

53. A Geobacillus or Ureibacillus as claimed in claim 44, wherein the enzyme which catalyses reaction I is acetyl-CoA acetyltransferase.

54. A Geobacillus or Ureibacillus as claimed in claim 44, wherein the enzyme which catalyses reaction II is 3-hydroxy-butyryl-CoA dehydrogenase.

55. A Geobacillus or Ureibacillus as claimed in claim 44, wherein the enzyme which catalyses reaction III is 3-hydroxy-butyryl-CoA dehydratase or crotonase.

56. A Geobacillus or Ureibacillus as claimed in claim 44 wherein the enzyme which catalyses reaction IV is butyryl-CoA dehydrogenase.

57. A Geobacillus or Ureibacillus as claimed in claim 44, wherein the enzyme which catalyses reaction V is butyraldehyde dehydrogenase or acetaldehyde dehydrogenase.

58. A Geobacillus or Ureibacillus as claimed in claim 44, wherein the enzyme which catalyses reaction VI is butanol dehydrogenase or acetaldehyde dehydrogenase.

59. A Geobacillus or Ureibacillus as claimed in claim 40, wherein the enzyme which catalyses reaction V or reaction VII is phosphate butyryltransferase.

60. A Geobacillus or Ureibacillus as claimed in claim 40, wherein the enzyme which catalyses reaction VI or VIII is butyrate kinase.

61. A method as claimed in claim 3, wherein:
(A) the Bacillaceae does not contain a functional lactate dehydrogenase gene or which does not produce lactate dehydrogenase or which produces a lower level of lactate dehydrogenase compared to a corresponding non-modified Bacillaceae,
(B) the Bacillaceae does not produce lactate or only produces trace amounts of lactate or which produces a lower level of lactate compared to a corresponding non-modified Bacillaceae,
(C) the Bacillaceae does not contain a functional ethanol dehydrogenase gene or which does not produce ethanol dehydrogenase or which produces a lower level of ethanol dehydrogenase compared to a corresponding non-modified Bacillaceae, or
(D) the Bacillaceae does not produce ethanol or only produces trace amounts of ethanol or which produces a lower level of ethanol compared to a corresponding non-modified Bacillaceae.

62. A recombinant Geobacillus or Ureibacillus as claimed in claim 44, wherein:
(A) the Geobacillus or Ureibacillus does not contain a functional lactate dehydrogenase gene or which does not produce lactate dehydrogenase or which produces a lower level of lactate dehydrogenase compared to a corresponding non-modified Geobacillus or Ureibacillus,
(B) the Geobacillus or Ureibacillus does not produce lactate or only produces trace amounts of lactate or which produces a lower level of lactate compared to a corresponding non-modified Geobacillus or Ureibacillus,
(C) the Geobacillus or Ureibacillus does not contain a functional ethanol dehydrogenase gene or which does not produce ethanol dehydrogenase or which produces a lower level of ethanol dehydrogenase compared to a corresponding non-modified Geobacillus or Ureibacillus, or
(D) the Geobacillus or Ureibacillus does not produce ethanol or only produces trace amounts of ethanol or which produces a lower level of ethanol compared to a corresponding non-modified Geobacillus or Ureibacillus.

63. A recombinant Geobacillus as claimed in claim 44, wherein the Geobacillus is Geobacillus thermoglucosidasius, Geobacillus kaustophilus, Geobacillus subterraneus, Geobacillus uzenensis, Geobacillus thermocatenulatus, Geobacillus thermodenitrificans, Geobacillus thermoleovorans, Geobacillus thermantarticus, Geobacillus pallidus, Geobacillus toebii, Geobacillus caldoxylosilyticus, Geobacillus tropicalis or Geobacillus stearothermophilus.

64. A recombinant Geobacillus as claimed in claim 63, wherein the Geobacillus is Geobacillus thermoglucosidasius, Geobacillus stearothermophilus or Geobacillus thermodenitrificans.

65. A process of producing butanol and/or butyrate comprising culturing a recombinant Geobacillus or Ureibacillus as claimed in claim 38, together with an appropriate substrate, and harvesting butanol and/or butyrate from the culture medium.

66. A process of producing butanol and/or butyrate comprising culturing a recombinant Geobacillus or Ureibacillus as claimed in claim 40, together with an appropriate substrate, and harvesting butanol and/or butyrate from the culture medium.

67. A process of producing butanol and/or butyrate comprising culturing a recombinant Geobacillus or Ureibacillus as claimed in claim 44, together with an appropriate substrate, and harvesting butanol and/or butyrate from the culture medium.
